(19) United States
(12) Patent Application Publication
(10) Pub. No.: US 2025/0002496 A1
(43) Pub. Date: Jan. 2, 2025

(54) INDOLE DERIVATIVES AS A MATRIX DESIGN FRAMEWORK FOR MALDI MASS SPECTROMETRY WITH BROAD APPLICATIONS

(71) Applicants: THE UAB RESEARCH FOUNDATION, BIRMINGHAM, AL (US); The Board of Trustees of the University of Alabama, Tuscaloosa, AL (US)

(72) Inventors: Gayan B. Wijeratne, Birmingham, AL (US); Pritam Mondal, Birmingham, AL (US); Qiaoli Liang, Tuscaloosa, AL (US)

(21) Appl. No.: 18/748,514

(22) Filed: Jun. 20, 2024

Related U.S. Application Data
(60) Provisional application No. 63/590,988, filed on Oct. 17, 2023.
(60) Provisional application No. 63/509,062, filed on Jun. 20, 2023.

(51) Int. Cl. C07D 487/04; G01N 33/68

(52) U.S. Cl. CPC C07D 487/04; G01N 33/6851

(57) ABSTRACT

The present disclosure provides for matrix materials for Matrix Assisted Laser Desportion/Ionization (MALDI) that include at least one MALDI matrix compound and methods of using the matrix materials, where the MALDI matrix compounds can be a derivative of indole.

### CROSS-REFERENCE TO RELATED APPLICATIONS

This application claims the benefit of U.S. Provisional Application entitled “INDOLE DERIVATIVES AS A MATRIX DESIGN FRAMEWORK FOR MALDI MASS SPECTROMETRY WITH BROAD APPLICATIONS” and having Ser. No. 63/509,062, filed Jun. 20, 2023, and this application claims the benefit of U.S. Provisional Application entitled “INDOLE DERIVATIVES AS A MATRIX DESIGN FRAMEWORK FOR MALDI MASS SPECTROMETRY WITH BROAD APPLICATIONS” and having Ser. No. 63/590,988, filed Oct. 17, 2023, each of which is herein incorporated by reference in their entireties.

### STATEMENT ON FUNDING PROVIDED BY THE U.S. GOVERNMENT

This invention was made with Government support under contract CHE-2045005 awarded by the National Science Foundation. The Government has certain rights in the invention.

### BACKGROUND

Matrix-Assisted Laser Desorption Ionization Mass Spectrometry (MALDI MS) has been applied to identify/detect a wide range of natural or synthetic compounds. MALDI matrix molecules absorb photons and transfer energy to the analyte and assist with analyte desorption and ionization. New MALDI matrix molecules are needed to further expand applications and improve analytical figures of merit. As matrix discoveries have often been by trial and error, identifying a matrix design framework is especially helpful to speed up new matrix development.

### SUMMARY

The present disclosure provide for matrix materials for Matrix Assisted Laser Desportion/Ionization (MALDI) that include at least one MALDI matrix compound and methods of using the matrix materials, where the MALDI matrix compounds can be a derivative of indole.

The present disclosure provides for a matrix material for Matrix Assisted Laser Desorption/Ionization (MALDI) comprising at least one MALDI matrix compound having a formula represented by the following structure:

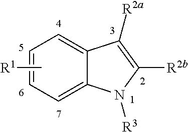

wherein each R¹ is independently selected from hydrogen, an alkyl group, an alkenyl group, an alkoxyl group, a carbonyl group, a carboxyl group, a primary amine group, a secondary amine group, a tertiary amine group, an ether group, halogen, hydroxy group, a cyano group, a thiol group, and a trifluoromethyl group; R^{2a} and R^{2b} are independently selected from hydrogen, an alkyl group, an alkenyl group, an alkoxyl group, a carbonyl group, a carboxyl group, a primary amine group, a secondary amine group, a tertiary amine group, an ether group, halogen, a hydroxy group, a cyano group, a thiol group, a trifluoromethyl group, and a substituted or unsubstituted indole group; and R³ is hydrogen or a substituted or unsubstituted indole group.

In an aspect, the present disclosure provides for at least one MALDI matrix compound having a formula represented by any one of the following structures:

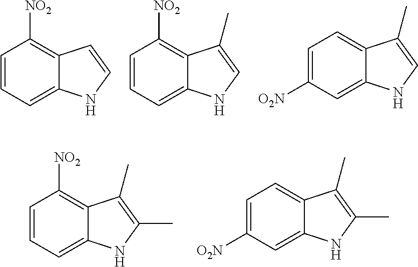

The present disclosure provides for a method for providing a plurality of analyte ions for Matrix Assisted Laser Desorption/Ionization (MALDI) mass spectrometry comprising: providing a matrix material comprising the at least one MALDI matrix compounds as described above and herein; depositing at least the matrix mixture and an analyte onto a support forming a matrix and analyte surface; irradiating the matrix and analyte surface to desorb and ionize at least part of the analyte into the gas-phase, forming a plurality of gas-phase ions; and separating the ionized analyte molecules based on their mass-to-charge ratio (m/z) using a mass analyzer.

The present disclosure provides for a method of nitrating indole, comprising: cooling a mixture comprising an iron porphyrin complex and a solvent; bubbling dioxygen gas through the mixture; adding an indole substrate to the mixture; and bubbling nitric oxide gas into the mixture. Also, the present disclosure provides for compounds produced using this method.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1A illustrates chemical structures of NI and FIG. 1B illustrates their UV-vis spectra (in methanol at room temperature) in comparison with common matrices.

FIG. 2 illustrates MALDI MS spectra of 3,4-MNI matrix (50 mM in ACN/water 50/50) in positive and negative ion modes with proposed structures for 2m−2H and 2m−4H dimers.

FIGS. 3A-3D illustrate PFOS MALDI MS quantitation acquired with 10 ppb ¹³C₈-PFOS IS and 3,4-MNI matrix in negative ion mode: FIG. 3A) MQ water PFOS calibration curve (0.05-10 ppb) and SEM image of the MQ water PFOS 10 ppb sample spot; FIG. 3B) tap water PFOS calibration curve (0.5-50 ppb) and SEM image of the tap water PFOS 10 ppb sample spot; FIG. 3C) MS spectra comparison of PFOS 0.5 ppb in tap and MQ water; FIG. 3D) tap water PFOS [M−H]⁻ ion intensity comparison with NaCl at 0, 100 mM, 500 mM, and 1M.

FIG. 4 illustrates bovine milk protein MS spectra acquired with 3,4-MNI, CHCA, 1,5-DAN, DHB, and SA matrices in positive ion mode.

FIGS. 5A-5D illustrate egg lipids MALDI MS spectra acquired with (FIG. 5A) 3,4-MNI in positive mode, (FIG. 5B) 3,4-MNI in negative mode, (FIG. 5C) DHB, 1,5-DAN, 3,4-MNI, 3,6-MNI, 2,3,4-DMNI, 2,3,6-DMNI in positive mode, and (FIG. 5D) 9-AA, 5-DAN, 3,4-MNI, 3,6-MNI, 2,3,4-DMNI, 2,3,6-DMNI in negative mode.

FIGS. 6A-6D illustrate blueberry crude methanol extract MALDI MS spectra acquired with (FIG. 6A) 2,3,6-DMNI in negative mode, (FIG. 6B) 3,6-MNI in positive mode, (FIG. 6C) 9-AA, 1,5-DAN, 3,4-MNI, 3,6-MNI, 2,3,4-DMNI, 2,3,6-DMNI in negative mode, and (FIG. 6D) DHB, 1,5-DAN, 3,4-MNI, 3,6-MNI, 2,3,4-DMNI, 2,3,6-DMNI in positive mode.

FIGS. 7A-7B illustrate blueberry metabolites/lipids MALDI imaging ion images acquired with 2,3,6-DMNI matrix in (FIG. 7A) negative and (FIG. 7B) positive ion mode, with m/z values listed above the images.

FIG. 8 illustrates an ¹H NMR spectrum (in CDCl3 at 298 K) of 3-Methyl-4-Nitro-1H-Indole (3,4-MNI): δ=8.36 (br, 1H), 7.81 (d, 1H), 7.60 (d, 1H), 7.22-7.18 (m, 2H), 2.41 (s, 3H).

FIG. 9 illustrates an ¹H NMR spectrum (in CDCl₃ at 298 K) of 3-Methyl-6-Nitro-1H-Indole (3,6-MNI): δ=8.41 (br, 1H), 8.33 (d, 1H), 8.02 (dd, 1H), 7.60 (d, 1H), 7.28 (m, 1H), 2.36 (s, 3H).

FIG. 10 illustrates an ¹H NMR spectrum (in CDCl₃ at 298 K) of 2,3-Dimethyl-4-Nitro-1H-Indole (2,3,4-DMNI): δ=8.28 (br, 1H), 7.75 (d, 1H), 7.51 (d, 1H), 7.11 (t, 1H), 2.44 (s, 3H), 2.29 (s, 3H).

FIG. 11 illustrates an ¹H NMR spectrum (in CDCl₃ at 298 K) of 2,3-Dimethyl-6-Nitro-1H-Indole (2,3,6-DMNI): δ=8.34 (br, 1H), 8.24 (d, 1H), 8.01 (dd, 1H), 7.48 (d, 1H), 2.46 (s, 3H), 2.26 (s, 3H).

FIG. 12 illustrates an ¹H NMR spectrum (in CDCl₃ at 298 K) of 4-Nitro-1H-Indole (4-NI): δ=8.54 (br, 1H), 8.09 (d, 1H), 7.65 (d, 1H), 7.42 (t, 1H), 7.26 (m, 1H), 7.21 (t, 1H).

FIG. 13 illustrates matrix detection sensitivity comparison for a) PC 16:0/16:0 [M+H]⁺ m/z 734.5 and [M+Na]⁺ m/z 756.5, b) PA 16:0/18:1 [M−H]⁻ m/z 673.5, c) PE 16:0/18:1 [M−H]⁻ m/z 716.5, and d) PI Glycine max major ion [M−H]⁻ m/z 833.5. Generally, 1 μL of matrix (1 and 5 mM in methanol) was applied onto a Bruker Anchorchip target and dried, followed by 1 μL of sample methanol solution applied on top. The lowest concentration spectra with S/N≥5 were listed in the figure. In b) PA_10 μM_4-NI_5 mM and PA 10 μM_CHCA_5 mM spectra, the observed masses were 0.2 Da higher than the expected m/z 673.5 Da (mass accuracy within 0.1 Da), and thus considered as part of the background ions.

FIG. 14 illustrates matrix detection sensitivity comparison for e) FibB [M+H]⁺ m/z 1552.7, f) FibB [M−H]⁻ m/z 1550.5, g) BSA [M+H]⁺ m/z 66.7 KDa & [M+2H]²⁺ m/z 33.4 KDa, and h) b-CN [M+H]⁺ m/z 24.0 KDa & [M+2H]²⁺ m/z 12.0 KDa. For FibB and BSA, 1 μL of matrix (5 mM or 25 mM in ACN/water 50/50 0.1% TFA) was applied onto a Bruker Anchorchip target and dried, followed by 1 μL of sample (in MQ water for FibB, in MQ water or 5 mM ammonium hydrogen citrate (AC) ACN/water 1/1 for BSA) applied on top. For b-CN protein, 5 μL of matrix (25 mM in ACN/water 50/50 0.1% TFA) was mixed with 1.5 μL sample (in MQ water or 5 mM ammonium hydrogen citrate ACN/water 50/50 0.1% TFA), with 1 μL applied onto a Bruker Anchorchip target and dried. The lowest concentration sample spectra with S/N≥5 were listed in the figure.

FIG. 15 illustrates matrix detection sensitivity comparison for i) LNDFHI [M+Na]⁺ m/z 1022.4, j) PFOS in MQ water [M−H]⁻ m/z 498.9, and k) PFOS in tap water [M−H]⁻ m/z 498.9. 1 μL of matrix (1 mM in methanol) was applied onto a Bruker Anchorchip target and dried, followed by 1 μL of sample (in MQ water for LNDFHI glycan, in MQ water or tap water for PFOS) applied on top. The lowest concentration sample spectra with S/N≥5 were listed in the figure. DHB 1 mM spectra (j) showed matrix background ion interference at the expected mass of PFOS.

FIG. 16 illustrates photo scanned images of matrix and sample crystalline formation on an Anchorchip target (a) and SEM images of the 3,4-MNI column spots 1-12 (m_5 mM refers to matrix 5 mM). For row 9, BSA 1 μM sample was prepared in 5 mM ammonium hydrogen citrate (50/50 ACN/water) for 3,4-MNI, 3,6-MNI, 2,3,4-DMNI, 2,3,6-DMNI, 4-NI, CHCA, and in MQ water for DHB, 9-AA, 1,5-DAN, SA, to correlate with the most sensitive sample preparation conditions. Row 1 (matrix blank in methanol) and row 7 (matrix blank in ACN/water 1/1 0.1% TFA) comparison demonstrated that the matrix crystalline formation on Anchorchip target was solvent dependent.

FIG. 17 illustrates MALDI MS spectra of 3,6-MNI matrix (50 mM in ACN/water 50/50) in positive and negative ion modes.

FIG. 18 illustrates MALDI MS spectra of 2,3,4-DMNI matrix (50 mM in ACN/water 50/50) in positive and negative ion modes.

FIG. 19 illustrates MALDI MS spectra of 2,3,6-DMNI matrix (50 mM in ACN/water 50/50) in positive and negative ion modes.

FIG. 20 illustrates MALDI MS spectra of 4-NI matrix (50 mM in ACN/water 50/50) in positive and negative ion modes.

FIG. 21 illustrates MALDI MS spectra of oligonucleotide 5′-ATGCGGAT-3′ acquired with 3,4-MNI matrix. Oligonucleotide standard 100 pmol/μL in MQ water was mixed with 3,4-MNI 50 mM in methanol and 100 mM ammonium monobasic phosphate in 50/50 methanol/water at 1:3:1 volume ratio, and 1 μL applied to the Anchorchip target. Observed ions included singly and doubly charged precursor ions [M−H]⁻[M−2H+K]⁻, [M−2H]²⁻, [M−3H+K]²⁻ and in-source-decay (ISD) fragment ions d₃, d₅, d₅+K, a₄-B, a₅-B, a₆-B, w₄. The ISD fragment ions were assigned following the oligonucleotide fragmentation nomenclature introduced by McLuckey, where aₙ-B means aₙ cleavage with a nucleotide base loss. (Reference: McLuckey, S. A.; Van Berkel, G. J.; Glish, G. L., Tandem mass spectrometry of small, multiply charged oligonucleotides. J. Am. Soc. Mass. Spectrom. 1992, 3 (1), 60-70.)

FIG. 22 illustrates MALDI MS spectra of polymer standards acquired with 3.4-MNI matrix: a) Tween 80 [M+Na]⁺ ions with a C₂H₄O repeating unit (44 Da), b) PMMA5k [M+Na]⁺ ions with a C₅H₈O₂ repeating unit (100 Da), and c) PS6000 [M+Ag]⁺ ions with a C₈H₈ repeating unit (104 Da). Tween 80 1.0 mg/mL in water and PMMASK 1.0 mg/mL in THF were mixed with 3,4-MNI (50 mM in methanol) at 1:2 and 1 μL applied to a stainless-steel target. PS6000 5 mg/mL in THF was mixed with 10 mg/mLAgTFA in THF at 2:1. One μL of the PS6000/AgTFA solution was spotted onto a stainless-steel target and dried, and one μL 3,4-MNI matrix (50 mM in ACN/water 85/15) was applied on top.

FIG. 23 illustrates QCM electrodes with NI matrices optical image comparison before and after admission into the repifleX MALDI vacuum at 2.0 e-7 Torr for 4 hours.

FIGS. 24A and 24B illustrates a comparison of MALDI MS spectra acquired with 3,4-MNI matrix: FIG. 24A) PFOS 0.05 ppb and 0 ppb background (10 ppb IS) in MQ water, and FIG. 24B) PFOS 0.5 ppb and 0 ppb background (10 ppb IS) in tap water.

FIG. 25 illustrates egg lipids MALDI TOF/TOF positive ion CID MS/MS spectra acquired with 3,4-MNI matrix for m/z a) 703.5 SM 16:0 [M+H]⁺ and b) 725.5 SM 16:0 [M+Na]⁺. a) The SM (16:0) [M+H]⁺ fragmentation was dominated with the SM head group ions m/z 184 [C₅H₁₄PO₄NH]⁺. b) The [M+Na]⁺ spectrum showed ions with loss of 59 [M+Na-NC₃H₉]⁺ and loss of 183 [M+Na-C₅H₁₄PO₄N]⁺.

FIG. 26 illustrates egg lipids MALDI TOF/TOF positive ion CID MS/MS spectra acquired with 3,4-MNI matrix for m/z a) 760.5 PC 34:1 [M+H]⁺, b) 782.5 PC 34:1 [M+Na]⁺, and c) 798.5 PC 34:1 [M+K]⁺. a) The PC 34:1 [M+H]⁺ spectrum showed fragment ions m/z 184.1 (PC head group, [C₅H₁₄PO₄NH]⁺), 478.4 [M+H-C₁₈H₃₄O₂]⁺, 496.4 [M+H-C₁₈H₃₂O]⁺, 504.4 [M+H⁻ C₁₆H₃₂O₂]⁺, 522.4 [M+H-C₁₆H₃₀O]⁺, 577.5 [M+H-C₅H₁₄PO₄N]⁺. b) The [M+Na]⁺ spectrum showed fragment ions m/z 723.5 [M+Na-NC₃H₉]⁺, 599.5 [M+Na-C₅H₁₄PO₄N]⁺, 577.5 [M+H-C₅H₁₄PO₄N]⁺, 496.4 [M+H-C₁₈H₃₂O]^{˜}, and m/z 146.9 [C₂H₅PO₄Na]⁺. c) The [M+K]⁺ spectrum showed fragment ions m/z 760.5 [M+K-K+H]⁺, m/z 739.5 [M+K-NC₃H₉]⁺, 577.5 [M+H-C₅H₁₄PO₄N]⁺, 496.4 [M+H-C₁₈H₃₂O]⁺, m/z 163.0 [C₂H₅PO₄K]⁺, and m/z 38.9 K⁺. Sodium adducted fragment ions (m/z 723.5 [M+Na-NC₃H₉]⁺, 599.5 [M+Na-C₅H₁₄PO₄N]⁺, and m/z 146.9 [C₂H₅PO₄Na]⁺) were also present in the CID spectra of m/z 798.5, likely due to sodium ion presence in the mass spectrometer gas phase.

FIG. 27 illustrates egg lipids MALDI TOF/TOF positive ion CID MS/MS spectra acquired with 3,4-MNI matrix for m/z a) 788.6 PC 36:1 [M+H]⁺, b) 810.6 PC 36:1 [M+Na]⁺/PC 38:4 [M+H]⁺, and c) 826.6 PC 36:1 [M+K]⁺. a) The PC 36:1 [M+H]⁺ spectrum showed major fragments m/z 184.1 (PC head group, [C₅H₁₄PO₄NH]⁺), 504.3 [M+H-C₁₈H₃₆O₂]⁺, 522.4 [M+H-C₁₈H₃₄O]⁺, 524.4 [M+H-C₁₈H₃₂O]^{˜}, 605.5 [M+H− 183]⁺. b) The [M+Na]⁺ spectrum showed PC 36:1 [M+Na]⁺ fragment ions m/z 751.4 [M+Na-NC₃H₉]⁺, 627.3 [M+Na-C₅H₁₄PO₄N]⁺, 146.9 [C₂H₅PO₄Na]⁺, 524.4 [M+H-C₁₈H₃₂O]⁺, and PC 38:4 [M+H]⁺ fragment ions 506.2 [M+H-C₂₀H₃₂O₂]⁺, 524.2 [M+H-C₂₀H₃₀O]⁺. C) The [M+K]⁺ spectrum showed fragment ions m/z 38.9 K⁺, 162.9 [C₂H₅PO₄K]⁺, 184.1, 524.0 [M+H-C₁₈H₃₂O]⁺, 605.5 [M+H− 183]⁺, and 767.4 [M+K NC₃H₉]⁺. The unidentified fragment ions might be from other type of ions within the ±2.0 Da precursor isolation window.

FIG. 28 illustrates egg lipids MALDI TOF/TOF negative ion CID MS/MS spectra acquired with 3,4-MNI matrix for m/z a) 671.5 PA 34:2, b) 673.5 PA 34:1, c) 687.5 PA 35:1, d) 697.5 PA 36:3, and e) 699.5 PA 36:2. PA head group fragment ions m/z PO₃⁻ 78.9, H₂PO₄ 96.9, C₃H₆PO₅⁻ 153.0 were observed in a)-d). Other ions assigned included a) PA 34:2 ions m/z 255.2 C₁₆H₃₁O₂⁻, 279.2 C₁₈H₃₁O₂⁻, 391.2 [M−H-C₁₈H₃₂O₂]⁻, 409.2 [M−H-C₁₈H₃₀O]⁻, 415.2 [M−H-C₁₆H₃₂O₂]⁻; b) PA 34:1 ions m/z 255.2 C₁₆H₃₁O₂⁻, 281.2 C₁₈H₃₃O₂⁻, 391.2 [M−H-C₁₈H₃₄O₂]⁻, 409.2 [M−H-C₁₈H₃₂O]⁻, 417.2 [M−H-C₁₆H₃₂O₂]⁻, and 435.2 [M−H-C₁₆H₃₀O]⁻; c) PA 35:1 ions m/z 255.2 C₁₆H₃₁O₂⁻, 281.2 C₁₈H₃₃O₂⁻, 391.2 [M−H-C₁₉H₃₆O₂]⁻, 405.2 [M−H-C₁₈H₃₄O₂]⁻, and 423.2 [M−H-C₁₈H₃₂O]⁻; d) PA 36:3 ions m/z 255.2 C₁₆H₃₁O₂⁻, 279.2 C₁₈H₃₁O₂⁻, 391.2 [M−H-C₂₀H₃₄O₂]⁻, 417.2 [M−H-C₁₈H₃₂O₂]⁻, e) PA 36:2 ions m/z 255.2 C₁₆H₃₁O₂⁻, 281.2 C₁₈H₃₃O₂⁻, 391.2 [M−H-C₂₀H₃₆O₂]⁻, 417.2 [M−H-C₁₈H₃₄O₂]⁻.

FIG. 29 illustrates egg lipids MALDI TOF/TOF negative ion CID MS/MS spectra acquired with 3,4-MNI matrix for m/z a) 714.5 PE 34:2, b) 716.5 PE 34:1, c) 738.5 PE 36:4, d) 740.5 PE 36:3, and e) 742.5 PE 36:2. Characteristic PE head group fragment ions are observed in a)-e): PO₃⁻ 78.9, H₂PO₄⁺ 96.9, C₃H₆PO₅⁻ 153.0, C₂H₅NPO₃⁺122.0, C₂H₇NPO₄⁻ 140.0. Other assigned ions: a) PE 34:2 ions m/z 255.2 C₁₆H₃₁O₂⁻, 279.2 C₁₈H₃₁O₂⁻, 452.3 [M−H-C₁₈H₃₀O]⁻, and 476.3 [M−H-C₁₆H₃₀O]⁻; b) PE 34:1 ions m/z 255.2 C₁₆H₃₁O₂⁻, 281.2 C₁₈H₃₃O₂⁻, 452.3 [M−H-C₁₈H₃₂O]⁻, and 478.3 [M−H-C₁₆H₃₀O]⁻; C) PE 36:4 ions m/z 255.2 C₁₆H₃₁O₂⁻, 279.2 C₁₈H₃₁O₂⁺, 303.2 C₂₀H₃₁O₂⁻, 452.3 [M−H-C₂₀H₃₀O]⁻, and 482.3 [M−H-C₁₆H₃₂O₂]⁻; d) PE 36:3 ions m/z 255.2 C₁₆H₃₁O₂⁻, 279.2 C₁₈H₃₁O₂⁻, 281.2 C₁₈H₃₃O₂⁻, 305.2 C₂₀H₃₃O₂⁻, 452.3 [M−H-C₂₀H₃₂O]⁻, and 478.3 [M−H-C₁₈H₃₀O]⁻; e) PE 36:2 ions m/z 255.2 C₁₆H₃₁O₂⁻, 279.2 C₁₈H₃₁O₂⁻, 283.2 C₁₈H₃₁O₂⁻, 307.2 C₂₀H₃₅O₂⁻, 458.3 [M−H-C₁₈H₃₆O₂]⁻, and 480.3 [M−H-C₁₈H₃₀O]⁻.

FIG. 30 illustrates egg lipids MALDI TOF/TOF negative ion CID MS/MS spectra acquired with 3,4-MNI matrix for m/z f) 744.5 PE 36:1, g) 762.5 PE 38:6, h) 764.5 PE 38:5, i) 766.5 PE 38:4, and j) 768.5 PE 38:3. Characteristic PE head group fragment ions are observed in f)-j): PO₃⁻ 78.9, H₂PO₄⁻ 96.9, C₃H₆PO₅⁺ 153.0, C₂H₅NPO₃⁺ 122.0, C₂H₇NPO₄⁻ 140.0. Other assigned ions: f) PE 36:1 ions m/z 281.2 C₁₈H₃₃O₂⁻, 283.2 C₁₈H₃₅O₂⁻, and 480.3 [M−H-C₁₈H₃₂O]⁻; g) PE 38:6 ions m/z 255.2 C₁₆H₃₁O₂⁻, 279.2 C₁₈H₃₁O₂⁻, 303.1 C₂₀H₃₁O₂⁻, 327.2 C₂₂H₃₁O₂⁻, 452.3 [M−H-C₂₂H₃₀O]⁻, and 480.3 [M−H-C₁₈H₃₄O₂]⁻; h) PE 38:5 m/z 255.2 C₁₆H₃₁O₂⁻, 283.2 C₁₈H₃₅O₂⁻, 303.1 C₂₀H₃₁O₂⁻, 329.2 C₂₂H₃₃O₂⁻, 452.3 [M−H-C₂₂H₃₂O]⁻, and 480.3 [M−H-C₁₈H₃₆O₂]⁻; i) PE 38:4 ions m/z 283.2 C₁₈H₃₅O₂⁻, 303.2 C₂₀H₃₁O₂⁻, 480.3 [M−H-C₂₀H₃₀O]⁻, and 500.3 [M−H-C₁₈H₃₄O]⁻; j) PE 38:3 ions m/z 283.2 C₁₈H₃₅O₂⁻, 305.2 C₂₀H₃₃O₂⁻, and 480.3 [M−H-C₂₀H₃₂O]⁻.

FIG. 31 illustrates egg lipids MALDI TOF/TOF negative ion CID MS/MS spectra acquired with 3,4-MNI matrix for m/z k) 790.5 PE 40:6, l) 792.5 PE 40:5, m) 794.5 PE 40:4. Characteristic PE head group fragment ions are observed in k)-m): PO₃⁻ 78.9, H₂PO₄⁻ 96.9, C₃H₆PO₅⁻ 153.0, C₂H₅NPO₃⁻ 122.0, C₂H₇NPO₄⁻ 140.0. Other assigned ions: k) PE 40:6 ions m/z 283.2 C₁₈H₃₅O₂⁻, 327.2 C₂₂H₃₁O₂⁻, and 480.3 [M−H-C₂₂H₃₀O]⁻; I) PE 40:5 ions m/z 283.2 C₁₈H₃₅O₂⁻, 329.2 C₂₂H₃₃O₂⁻, and 480.3 [M−H-C₂₂H₃₂O]⁻; m) PE 40:4 m/z 283.2 C₁₈H₃₅O₂⁻, 331.2 C₂₂H₃₅O₂⁻, and 480.3 [M−H-C₂₂H₃₄O]⁻.

FIG. 32 illustrates egg lipids MALDI TOF/TOF negative ion CID MS/MS spectra acquired with 3,4-MNI matrix for m/z a) 833.5 PI 34:2, b) 835.5 PI 34:1, c) 857.5 PI 36:4, d) 859.5 PI 36:3, and e) 861.5 PI 36:2. Characteristic PI head group fragment ions m/z PO₃⁻ 78.9, H₂PO₄⁻ 96.9, C₃H₆PO₅⁻ 153.0, C₆H₈O₇P⁻ 223.0, C₆H₁₀O₈P⁻ 241.0 were observed in each spectrum a)-e). Other ions assigned: a) m/z 255.2 C₁₆H₃₁O₂⁻, 279.2 C₁₈H₃₁O₂⁻, 391.2 [M−H-C₁₈H₃₂O₂⁻ 162(inositol)]⁻, 553.3 [M−H-C₁₈H₃₂O₂]⁻, and 577.3 [M−H-C₁₆H₃₂O₂]⁻; b) m/z 255.2 C₁₆H₃₁O₂⁻, 281.2 C₁₈H₃₃O₂⁻, 391.2 [M−H-C₁₈H₃₄O₂− 162(inositol)]⁻, 553.3 [M−H-C₁₈H₃₄O₂]⁻, and 579.3 [M−H-C₁₆H₃₂O₂]⁻; c) m/z 255.2 C₁₆H₃₁O₂⁻, 279.2 C₁₈H₃₁O₂⁻, 303.1 C₂₀H₃₁O₂⁻, 391.2 [M−H-C₂₀H₃₂O₂⁻ 162(inositol)]⁻, 553.3 [M−H-C₂₀H₃₂O₂]⁻, and 601.3 [M−H-C₁₆H₃₂O₂]⁻; d) m/z 255.2 C₁₆H₃₁O₂⁻, 279.2 C₁₈H₃₁O₂⁻, 281.2 C₁₈H₃₃O₂⁻, 305.1 C₂₀H₃₃O₂⁻, 391.2 [M−H-C₂₀H₃₄O₂⁻ 162(inositol)]⁻, 553.3 [M−H-C₂₀H₃₄O₂]⁻, and 579.3 [M−H-C₁₈H₃₂O₂]⁻; e) m/z 255.2 C₁₆H₃₁O₂⁻, 279.2 C₁₈H₃₁O₂⁻, 283.2 C₁₈H₃₅O₂⁻, 307.1 C₂₀H₃₅O₂⁻, 391.2 [M−H-C₂₀H₃₆O₂⁻ 162(inositol)]⁻, 553.3 [M−H-C₂₀H₃₆O₂]⁻, and 581.3 [M−H-C₁₈H₃₂O₂]⁻.

FIG. 33 illustrates egg lipids MALDI TOF/TOF negative ion CID MS/MS spectra acquired with 3,4-MNI matrix for m/z f) 885.5 PI 38:4, g) 887.5 PI 38:3, h) 911.5 PI 40:5, and i) 913.5 PI 40:4. Characteristic PI head group fragment ions m/z PO₃⁻ 78.9, H₂PO₄⁻ 96.9, C₃H₆PO₅⁻ 153.0, C₆H₈O₇P⁻ 223.0, C₆H₁₀O₈P⁻ 241.0 are observed in each spectrum f)-i). Other ions assigned: f) m/z 283.2 C₁₈H₃₅O₂⁻, 303.2 C₂₀H₃₁O₂⁻, 419.2 [M−H-C₂₀H₃₂O₂⁻ 162(inositol)]⁻, 581.3 [M−H-C₂₀H₃₂O₂]⁻, and 619.3 [M−H-C₁₈H₃₄O]⁻; g) m/z 283.2 C₁₈H₃₅O₂⁻, 305.2 C₂₀H₃₃O₂⁻, 419.2 [M−H-C₂₀H₃₄O₂⁻ 162(inositol)]⁻, and 581.3 [M−H-C₂₀H₃₄O₂]⁻; h) m/z 283.2 C₁₈H₃₅O₂⁻, 303.2 C₂₀H₃₁O₂⁻, 329.2 C₂₂H₃₃O₂⁻, 419.2 [M−H-C₂₂H₃₄O₂⁻ 162(inositol)]⁻, 581.3 [M−H-C₂₂H₃₄O₂]⁻, and 607.3 [M−H-C₂₀H₃₂O₂]⁻; i) m/z 283.2 C₁₈H₃₅O₂⁻, 305.2 C₂₀H₃₃O₂⁻, 331.2 C₂₂H₃₅O₂⁻, 419.2 [M−H-C₂₂H₃₆O₂− 162(inositol)]⁻, 581.3 [M−H-C₂₂H₃₆O₂]⁻, and 607.3 [M−H-C₂₀H₃₄O₂]⁻.

FIG. 34 illustrates blueberry tissue MALDI MS spectra comparison in positive (a) and negative (b) mode under high (dried droplet, 80% absolute laser power) and low (imaging 50, 51% absolute laser power) laser power, with 2,3,6-DMNI matrix layer above tissue or under tissue or without matrix. For each combination, three spectra were taken at the bulk of the blueberry interior (mesocarp region) and the optimum spectra were used for comparison.

FIG. 35 illustrates blueberry tissue optical image comparison before and after vacuum (4 hour and 24 hour) with 2,3,6-DMNI matrix thin layer applied above tissue, under tissue, above & under tissue, and without matrix.

FIG. 36 illustrates Table 1. Detection sensitivity comparison of NI matrices with common matrices in positive ion (+) and negative ion (−) mode. PFOS and PI were tested at 10, 1, 0.1, 0.01, 0.001 ppm. All other compounds were tested at 10, 1, 0.1, 0.01, 0.001 μM, and the lowest concentration with S/N≥5 was recorded (x, S/N<5 at examined concentrations).

### DETAILED DESCRIPTION

The present disclosure provides for matrix materials for Matrix Assisted Laser Desportion/Ionization (MALDI) that include at least one MALDI matrix compound and methods of using the matrix materials, where the MALDI matrix compounds can be a derivative of indole.

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Prior to describing the various embodiments, the following definitions are provided and should be used unless otherwise indicated.

### Definitions

It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

It will be understood by those skilled in the art that the moieties substituted can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF3, —CN and the like. Cycloalkyls can be substituted in the same manner.

The term “acyl” as used herein, alone or in combination, means a carbonyl or thiocarbonyl group bonded to a radical selected from, for example, optionally substituted, hydrido, alkyl (e.g. haloalkyl), alkenyl, alkynyl, alkoxy (“acyloxy” including acetyloxy, butyryloxy, iso-valeryloxy, phenylacetyloxy, berizoyloxy, p-methoxybenzoyloxy, and substituted acyloxy such as alkoxyalkyl and haloalkoxy), aryl, halo, heterocyclyl, heteroaryl, sulfonyl (e.g. allylsulfinylalkyl), sulfonyl (e.g. alkylsulfonylalkyl), cycloalkyl, cycloalkenyl, thioalkyl, thioaryl, amino (e.g alkylamino or dialkylamino), and aralkoxy. Illustrative examples of “acyl” radicals are formyl, acetyl, 2-chloroacetyl, 2-bromacetyl, benzoyl, trifluoroacetyl, phthaloyl, malonyl, nicotinyl, and the like. The term “acyl” as used herein refers to a group —C(O)R₂₆, where R₂₆ is hydrogen, alkyl, cycloalkyl, cycloheteroalkyl, aryl, arylalkyl, heteroalkyl, heteroaryl, and heteroarylalkyl. Examples include, but are not limited to formyl, acetyl, cyclohexylcarbonyl, cyclohexylmethylcarbonyl, benzoyl, beozylcarbonyl and the like.

The term “alkyl”, either alone or within other terms such as “thioalkyl” and “arylalkyl”, as used herein, means a monovalent, saturated hydrocarbon radical which may be a straight chain (i.e. linear) or a branched chain. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like. An alkyl radical for use in the present disclosure generally comprises from about 1 to 20 carbon atoms, particularly from about 1 to 10, 1 to 8 or 1 to 7, more particularly about 1 to 6 carbon atoms, or 3 to 6. Illustrative alkyl radicals include methyl, ethyl, n-propyl, n-butyl, n-pentyl, n-hexyl, isopropyl, isobutyl, isopentyl, amyl, sec-butyl, tert-butyl, tert-pentyl, n-heptyl, n-actyl, n-nonyl, n-decyl, undecyl, n-dodecyl, n-tetradecyl, pentadecyl, n-hexadecyl, heptadecyl, n-octadecyl, nonadecyl, eicosyl, dosyl, n-tetracosyl, and the like, along with branched variations thereof. In certain aspects of the disclosure an alkyl radical is a C1-C6 lower alkyl comprising or selected from the group consisting of methyl, ethyl, n-propyl, n-butyl, n-pentyl, n-hexyl, isopropyl, isobutyl, isopentyl, amyl, tributyl, sec-butyl, tert-butyl, tert-pentyl, and n-hexyl. An alkyl radical may be optionally substituted with substituents as defined herein at positions that do not significantly interfere with the preparation of compounds of the disclosure and do not significantly reduce the efficacy of the compounds. In certain aspects of the disclosure, an alkyl radical is substituted with one to five substituents including halo, lower alkoxy, lower aliphatic, a substituted lower aliphatic, hydroxy, cyano, nitro, thio, amino, keto, aldehyde, ester, amide, substituted amino, carboxyl, sulfonyl, sulfuryl, sulfenyl, sulfate, sulfoxide, substituted carboxyl, halogenated lower alkyl (e.g. CF₃), halogenated lower alkoxy, hydroxycarbonyl, lower alkoxycarbonyl, lower alkylcarbonyloxy, lower alkylcarbonylamino, cycloaliphatic, substituted cycloaliphatic, or aryl (e.g., phenylmethyl benzyl)), heteroaryl (e.g., pyridyl), and heterocyclic (e.g., piperidinyl, morpholinyl). Substituents on an alkyl group may themselves be substituted.

The terms “alkoxyl” or “alkoxyalkyl” as used herein refer to an alkyl-O— group wherein alkyl is as previously described. The term “alkoxyl” as used herein can refer to C₁₋₂₀ inclusive, linear, branched, or cyclic, saturated or unsaturated oxo-hydrocarbon chains, including, for example, methoxyl, ethoxyl, propoxyl, isopropoxyl, butoxyl, t-butoxyl, and pentoxyl

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkenyl” as used herein is a hydrocarbon group of from 2 to 24 carbon atoms or 2 to 8 carbon atoms or 2 to 6 carbon atoms with a structural formula containing at least one carbon-carbon double bond. Asymmetric structures such as (R¹R²)C═C(R³R⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

As used herein, “alkynyl” or “alkynyl group” refers to straight or branched chain hydrocarbon groups having 2 to 40, 2 to 20, 2 to 10, or 2 to 5 carbon atoms and at least one triple carbon to carbon bond, such as ethynyl. Reference to “alkynyl” or “alkynyl group” includes unsubstituted and substituted forms of the hydrocarbon moiety.

The term “cycloalkynyl” as used herein is a non-aromatic carbon-based ring composed of at least seven carbon atoms and containing at least one carbon-carbon triple bound. Examples of cycloalkynyl groups include, but are not limited to, cyclooctynyl, cyclononynyl, and the like. The term “heterocycloalkynyl” is a type of cycloalkenyl group as defined above and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The Ar (e.g., Ar₁, Ar₂, etc) group is an aromatic system or group such as an aryl group. “Aryl”, as used herein, refers to C₅-C₂₀-membered aromatic, heterocyclic, fused aromatic, fused heterocyclic, biaromatic, or bihetereocyclic ring systems. In an aspect, “aryl”, can include 5-, 6-, 7-, 8-, 9-, and 10-membered single-ring aromatic groups that may include from zero to four heteroatoms, for example, functional groups that correspond to benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein.

The term “aryl” also includes polycyclic ring systems (C₅-C₃₀) having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

In some aspects, a structure of a compound can be represented by a formula:

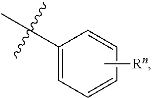

which is understood to be equivalent to a formula:

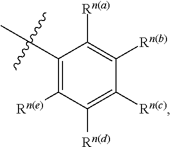

wherein n is typically an integer. That is, Rn is understood to represent five independent substituents, Rn(a), Rn(b), Rn(c), Rn(d), and Rn(e). By “independent substituents,” it is meant that each R substituent can be independently defined. For example, if in one instance Rn(a) is halogen, then Rn(b) is not necessarily halogen in that instance.

The term “carboxyl” as used herein, alone or in combination, refers to —C(O)OR₂₅— or —C(—O)OR₂₅ wherein R₂₅ is hydrogen, alkyl, alkenyl, alkynyl, cycloalkyl, cycloalkenyl, amino, thiol, aryl, heteroaryl, thioalkyl, thioaryl, thioalkoxy, a heteroaryl, or a heterocyclic, which may optionally be substituted. In aspects of the disclosure, the carboxyl groups are in an esterified form and may contain as an esterifying group lower alkyl groups. In particular aspects of the disclosure, —C(O)OR₂₅ provides an ester or an amino acid derivative. An esterified form is also particularly referred to herein as a “carboxylic ester”. In aspects of the disclosure a “carboxyl” may be substituted, in particular substituted with allyl which is optionally substituted with one or more of amino, amine, halo, alkylamino, aryl, carboxyl, or a heterocyclic. Examples of carboxyl groups are methoxycarbonyl, butoxycarbonyl, tert.alkoxycarbonyl such as tert-butoxycarbonyl, arylmethyoxycarbonyl having one or two aryl radicals including without limitation phenyl optionally substituted by for example lower alkyl, lower alkoxy, hydroxyl, halo, and/or nitro, such as benzyloxycarbonyl, methoxybenzyloxycarbonyl, diphenylmethoxycarbonyl, 2-bromoethoxycarbonyl, 2-iodoethoxycarbonyltert.butylcarborlyl, 4-nitrobenzyloxycarbonyl, diphenylmethoxycarbonyl, benzhydroxycarbonyl, di-(4-methoxyphenyl-methoxycarbonyl, 2-bromoethoxycarbonyl, 2-iodoethoxycarbonyl, 2-trimethylsilylethoxycarbonyl, or 2-triphenylsilylethoxycarbonyl. Additional carboxyl groups in esterified form are silyloxycarbonyl groups including organic silyloxycarbonyl. The silicon substituent in such compounds may be substituted with lower alkyl (e.g. methyl), alkoxy (e.g. methoxy), and/or halo (e.g. chlorine). Examples of silicon substituents include trimethylsilyi and dimethyltert.butylsilyl. In aspects of the disclosure, the carboxyl group may be an alkoxy carbonyl, in particular methoxy carbonyl, ethoxy carbonyl, isopropoxy carbonyl, t-butoxycarbonyl, t-pentyloxycarbonyl, sir heptyloxy carbonyl, especially methoxy carbonyl or ethoxy carbonyl. The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure.

The term “Matrix Assisted Laser Desoprtion/Ionization” or “MALDI” refers to an ionization technique that involves a laser striking a sample surface (comprising at least a MALDI matrix and an analyte) to desorb and/or ionize analyte molecules into the gas phase with or without fragmenting the molecules. In MALDI mass spectrometry, the ionization step is followed by an analysis of the mass-to-charge (m/z) ratio of the ionized analyte molecules using a mass analyzer and a detector. The mass analyzer may include any type of apparatus suitable for the separation of ions, including time of flight, quadrupole, magnetic sector, ion trap, orbitrap, fourier transform ion cyclotron resonance, any combination thereof, and the like.

Compounds of the disclosure can be prepared using reactions and methods generally known to the person of ordinary skill in the art, having regard to that knowledge and the disclosure of this application including the Examples. The reactions are performed in solvents appropriate to the reagents and materials used and suitable for the reactions being affected. It will be understood by those skilled in the art of organic synthesis that the functionality present on the compounds should be consistent with the proposed reaction steps. This will sometimes require modification of the order of the synthetic steps or selection of one particular process scheme over another in order to obtain a desired compound of the disclosure. It will also be recognized that another major consideration in the development of a synthetic route is the selection of the protecting group used for protection of the reactive functional groups present in the compounds described in this disclosure. An authoritative account describing the many alternatives to the skilled artisan is Greene and Wuts (Protective Groups In Organic Synthesis, Wiley and Sons, 1991).

### Discussion

The present disclosure provides for matrix materials for Matrix Assisted Laser Desportion/Ionization (MALDI) that include at least one MALDI matrix compound and methods of using the matrix materials. The MALDI matrix compounds can be compatible with MALDI in both positive and negative ion detection modes. The MALDI matrix compounds can be a derivative of indole, such as those provided herein.

In one aspect, the matrix material for MALDI can include at least one MALDI matrix compound with a formula represented by the following structure:

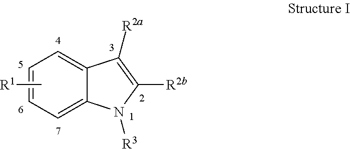

In one aspect, each R¹ group can be independently selected from hydrogen, a substituted or unsubstituted alkyl group (e.g., —CH₃, —C₂H₅, —C₃H₇, —C₄H₉, —CH₂COOH, —CH₂CONH₂, —CH₂OH, —CH₂NH₂, —CH₂N(CH₃)₂, —CH₂CN, or —CH₂CH(CN)₂), a substituted or unsubstituted alkenyl group (e.g., —C₂H₃, —C₃H₅, —C₄H₅, —C₂H₂COOH, —C₂H₂COOCH₃), an alkoxyl group (e.g., —OCH₃), a carbonyl group (e.g., —COH, —COCH₃, —CONH₂, —CONH(NH₂)), a carboxyl group (e.g., —COOH), a primary amine group, a secondary amine group (e.g., —NH(CH₃), —NH(NH₂), or —NH(C₂H₄(NH₂)), a tertiary amine group (e.g., —N(CH₃)₂), an ether group (e.g., —CH₂OCH₃), halogen, a hydroxy group, a cyano group, a nitro group, a thiol group, or a trifluoromethyl group. An R¹ group can be present on any of carbons 4-7. When two or more R¹ groups are present, each R¹ group can be present on the 4-7 carbons. When more than one R¹ group is present, the R¹ groups can be different, the same, or a mixture (e.g., 1 R¹ group is an alkyl, and 2 R¹ groups are a primary amine).

In another aspect, at least one R¹ group is nitro and the remaining R¹ groups can be independently selected from hydrogen, a substituted or unsubstituted alkyl group (e.g., —CH₃, —C₂H₅, —C₃H₇, —C₄H₉, —CH₂COOH, —CH₂CONH₂, —CH₂OH, —CH₂NH₂, —CH₂N(CH₃)₂, —CH₂CN, or —CH₂CH(CN)₂), a substituted or unsubstituted alkenyl group (e.g., —C₂H₃, —C₃H₅, —C₄H₅, —C₂H₂COOH, —C₂H₂COOCH₃), an alkoxyl group (e.g., —OCH₃), a carbonyl group (e.g., —COH, —COCH₃, —CONH₂, —CONH(NH₂)), a carboxyl group (e.g., —COOH), a primary amine group, a secondary amine group (e.g., —NH(CH₃), —NH(NH₂), or —NH(C₂H₄(NH₂)), a tertiary amine group (e.g., —N(CH₃)₂), an ether group (e.g., —CH₂OCH₃), halogen, a hydroxy group, a cyano group, a nitro group, a thiol group, or a trifluoromethyl group.

R^{2a} and R^{2b} can be independently selected from hydrogen, a substituted or unsubstituted alkyl group (e.g., —CH₃, —C₂H₅, —C₃H₇, —C₄H₉, —CH₂COOH, —CH₂CONH₂, —CH₂OH, —CH₂NH₂, —CH₂N(CH₃)₂, —CH₂CN, or —CH₂CH(CN)₂), a substituted or unsubstituted alkenyl group (e.g., —C₂H₃, —C₃H₅, —C₄H₅, —C₂H₂COOH, —C₂H₂COOCH₃), an alkoxyl group (e.g., —OCH₃), a carbonyl group (e.g., —COH, —COCH₃, —CONH₂, —CONH(NH₂)), a carboxyl group (e.g., —COOH), a primary amine group, a secondary amine group (e.g., —NH(CH₃), —NH(NH₂), or —NH(C₂H₄(NH₂)), a tertiary amine group (e.g., —N(CH₃)₂), an ether group (e.g., —CH₂OCH₃), halogen, a hydroxy group, a cyano group, a nitro group, a thiol group, or a trifluoromethyl group, or a substituted or unsubstituted indole group. R³ can be hydrogen or a substituted or unsubstituted indole group.

In another aspect, each R¹ group can be independently selected from hydrogen, a substituted or unsubstituted alkyl group (e.g., —CH₃, —C₂H₅, —C₃H₇, —C₄H₉, —CH₂COOH, —CH₂CONH₂, —CH₂OH, —CH₂NH₂, —CH₂N(CH₃)₂, —CH₂CN, or —CH₂CH(CN)₂), a substituted or unsubstituted alkenyl group (e.g., —C₂H₃, —C₃H₅, —C₄H₅, —C₂H₂COOH, —C₂H₂COOCH₃), an alkoxyl group (e.g., —OCH₃), a primary amine, a secondary amine (e.g., —NH(CH₃), —NH(NH₂), or —NH(C₂H₄(NH₂)), a tertiary amine (e.g., —N(CH₃)₂), an ether (e.g., —CH₂OCH₃), halogen, hydroxy, cyano, nitro, thiol, or trifluoromethyl. R^{2a} and R^{2b} can be independently selected from hydrogen, a substituted or unsubstituted alkyl group (e.g., —CH₃, —C₂H₅, —C₃H₇, —C₄H₉, —CH₂COOH, —CH₂CONH₂, —CH₂OH, —CH₂NH₂, —CH₂N(CH₃)₂, —CH₂CN, or —CH₂CH(CN)₂), a substituted or unsubstituted alkenyl group (e.g., —C₂H₃, —C₃H₅, —C₄H₅, —C₂H₂COOH, —C₂H₂COOCH₃), an alkoxyl group (e.g., —OCH₃), a primary amine, a secondary amine (e.g., —NH(CH₃), —NH(NH₂), or —NH(C₂H-₄(NH₂)), a tertiary amine (e.g., —N(CH₃)₂), an ether (e.g., —CH₂OCH₃), halogen, hydroxy, cyano, nitro, thiol, trifluoromethyl, or a substituted or unsubstituted indole group. R³ can be hydrogen or a substituted or unsubstituted indole group.

In another aspect, each R¹ group can be independently selected from hydrogen, a substituted or unsubstituted alkyl group (e.g., —CH₃, —C₂H₅, —C₃H₇, —C₄H₉, —CH₂COOH, —CH₂CONH₂, —CH₂OH, —CH₂NH₂, —CH₂N(CH₃)₂, —CH₂CN, or —CH₂CH(CN)₂), a substituted or unsubstituted alkenyl group (e.g., —C₂H₃, —C₃H₅, —C₄H₅, —C₂H₂COOH, —C₂H₂COOCH₃), an alkoxyl group (e.g., —OCH₃), a carbonyl group (e.g., —COH, —COCH₃, —CONH₂, —CONH(NH₂)), a carboxyl group (e.g., —COOH), hydroxy, cyano, or nitro. R^{2a} and R^{2b} can be independently selected from hydrogen, a substituted or unsubstituted alkyl group (e.g., —CH₃, —C₂H₅, —C₃H₇, —C₄H₉, —CH₂COOH, —CH₂CONH₂, —CH₂OH, —CH₂NH₂, —CH₂N(CH₃)₂, —CH₂CN, or —CH₂CH(CN)₂), a substituted or unsubstituted alkenyl group (e.g., —C₂H₃, —C₃H₅, —C₄H₅, —C₂H₂COOH, —C₂H₂COOCH₃), an alkoxyl group (e.g., —OCH₃), a carbonyl group (e.g., —COH, —COCH₃, —CONH₂, —CONH(NH₂)), a carboxyl group (e.g., —COOH), hydroxy, cyano, nitro, or a substituted or unsubstituted indole group. R³ can be hydrogen or a substituted or unsubstituted indole group.

In another aspect, at least one R¹ group is nitro and the remaining R¹ groups can be independently selected from hydrogen, a C1 to C6 alkyl group, a carbonyl group (e.g., —COH, —COCH₃, —CONH₂, —CONH(NH₂)), a carboxyl group (e.g., —COOH), nitro, thiol, a primary amine, a secondary amine (e.g., —NH(CH₃), —NH(NH₂), or —NH(C₂H₄(NH₂)), or a tertiary amine (e.g., —N(CH₃)₂). R^{2a} and R^{2b} can be independently selected from hydrogen, a C1 to C6 alkyl group, a carbonyl group (e.g., —COH, —COCH₃, —CONH₂, —CONH(NH₂)), a carboxyl group (e.g., —COOH), nitro, thiol, a primary amine, a secondary amine (e.g., —NH(CH₃), —NH(NH₂), or —NH(C₂H₄(NH₂)), a tertiary amine (e.g., —N(CH₃)₂), or a substituted or unsubstituted indole group. R³ can be a hydrogen or a substituted or unsubstituted indole group.

In some aspects, the substituent at position 4 of Structure I is a nitro group and the substituent at position 1 is a hydrogen. In other aspects, the substituent at position 4 of Structure I is a nitro group, the substituent at position 3 is a methyl group, and the substituent at position 1 is a hydrogen. In other aspects, the substituent at position 4 of Structure I is a nitro group, the substituents at positions 2 and 3 are each a methyl group, and the substituent at position 1 is a hydrogen. In other aspects, the substituent at position 6 of Structure I is a nitro group, the substituents at positions 2 and 3 are each a methyl group, and the substituent at position 1 is a hydrogen. In yet other aspects, the substituent at position 6 of Structure I is a nitro group, the substituent at position 3 is a methyl group, and the substituent at position 1 is a hydrogen.

In an aspect, the substituted or unsubstituted indole group substituent can have a formula represented by the following structure:

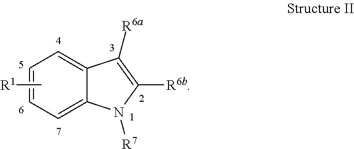

In some aspects, Structure II can be bonded at substitution position 1, 2, or 3 to Structure I as an R^{2a}, R^{2b}, or R³ substituent, where Structure I takes the place of substituent R⁷, R^{6b}, or R^{6a}, respectively. In some aspects, each R⁵ group can be independently selected from hydrogen, a substituted or unsubstituted alkyl group (e.g., —CH₃, —C₂H₅, —C₃H₇, —C₄H₉, —CH₂COOH, —CH₂CONH₂, —CH₂OH, —CH₂NH₂, —CH₂N(CH₃)₂, —CH₂CN, or —CH₂CH(CN)₂), a substituted or unsubstituted alkenyl group (e.g., —C₂H₃, —C₃H₅, —C₄H₅, —C₂H₂COOH, —C₂H₂COOCH₃), an alkoxyl group (e.g., —OCH₃), a carbonyl group (e.g., —COH, —COCH₃, —CONH₂, —CONH(NH₂)), a carboxyl group (e.g., —COOH), a primary amine, a secondary amine (e.g., —NH(CH₃), —NH(NH₂), or —NH(C₂H₄(NH₂)), a tertiary amine (e.g., —N(CH₃)₂), an ether (e.g., —CH₂OCH₃), halogen, hydroxy, cyano, nitro, thiol, or trifluoromethyl. An R⁵ group can be present on any of carbons 4-7. When two or more R⁵ groups are present, each R⁵ group can be present on the 4-7 carbons. When more than one R⁵ group is present, the R⁵ groups can be different, the same, or a mixture (e.g., 1 R⁵ group is an alkyl, and 2 R⁵ groups are a primary amine).

R^{6a} and R^{6b} can be independently selected from hydrogen, a substituted or unsubstituted alkyl group (e.g., —CH₃, —C₂H₅, —C₃H₇, —C₄H₉, —CH₂COOH, —CH₂CONH₂, —CH₂OH, —CH₂NH₂, —CH₂N(CH₃)₂, —CH₂CN, or —CH₂CH(CN)₂), a substituted or unsubstituted alkenyl group (e.g., —C₂H₃, —C₃H₅, —C₄H₅, —C₂H₂COOH, —C₂H₂COOCH₃), an alkoxyl group (e.g., —OCH₃), a carbonyl group (e.g., —COH, —COCH₃, —CONH₂, —CONH(NH₂)), a carboxyl group (e.g., —COOH), a primary amine, a secondary amine (e.g., —NH(CH₃), —NH(NH₂), or —NH(C₂H₄(NH₂)), a tertiary amine (e.g., —N(CH₃)₂), an ether (e.g., —CH₂OCH₃), halogen, hydroxy, cyano, nitro, thiol, or trifluoromethyl, or a substituted or unsubstituted indole group. R⁷ can be hydrogen or a substituted or unsubstituted indole group, similar to that represented by Structure II. R⁷ can be hydrogen or a substituted or unsubstituted indole group, similar to that represented by Structure II. In some aspects, the matrix material can be a substituted indole dimer, a substituted indole trimer, a substituted indole tetramer, a substituted indole pentamer, or a substituted indole hexamer. The matrix material can also include longer oligomer chains made up of substituted indoles.

In other aspects, the matrix material for MALDI can include at least one MALDI matrix compound with a formula represented by any one of the following structures:

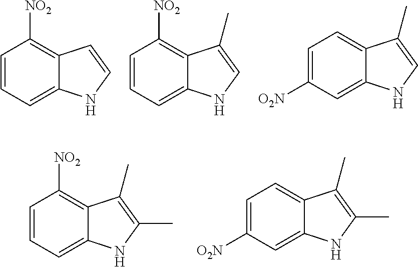

One aspect of this disclosure provides for a method of providing analyte ions for MALDI mass spectrometry. This method can include providing a matrix material including at least one MALDI matrix compound, depositing at least the matrix material and an analyte onto a support to form a matrix and analyte surface, irradiating the matrix and analyte surface desorb and ionize at least part of the analyte into the gas phase, forming gas-phase ions, and separating the ionized analyte molecules based on their mass-to-charge ratio using a mass analyzer. In a further aspect, the matrix material and analyte can be mixed together to form a first mixture, and the first mixture can be deposited onto the support to form the matrix and analyte surface. This first mixture can further include additives such as salts (e.g., NaCl, KI, AgNO₃), buffers (e.g., ammonium hydrogen citrate, ammonium monobasic phosphate, ammonium acetate), small molecules (e.g., trifluoro acetic acid, ammonia, HCl, H₃PO₄, fucose, fructose), or any combination thereof. In another aspect, the matrix material and analyte can be deposited by spotting the matrix material and analyte in layers onto the support. The process of spotting can further include adding additive layers, including additives such as those discussed previously. In one aspect, the order of the matrix, analyte, and, optionally, additive layers can vary. In another aspect, the matrix material can be sprayed or sublimed onto the support. The analyte can be deposited onto the support prior to the spraying or subliming or after the spraying or subliming. In a further aspect, the matrix material can further comprise additives, such as those mentioned above, that are also sprayed or sublimed onto the support. In other aspects, the analyte can be deposited onto the support in the form of a solution, suspension, solid particles, or surface layers. Any combination of methods for depositing the analyte and matrix material onto the support can also be performed.

In some aspects, the mass analyzer can be selected from time of flight, quadrupole, magnetic sector, ion trap, orbitrap, fourier transform ion cyclotron resonance, and any combination thereof. In addition to mass-to-charge ratio separation by a mass analyzer, the gas phase ions can be further separated by their mobility through a buffer gas (e.g., He, N₂, Ar, CO₂, N₂O). In some aspects, when there is analyte fragmentation, the analyte can undergo further structural characterization in the mass analyzer. The analyte can comprise a biomolecule, such as an amino acid, lipid, peptide, protein, glycan, carbohydrate, oligonucleotide, metabolite, environmental contaminant (e.g., per- and polyfluoroalkyl substances and pesticides), pharmaceutical, polymer, and the like. In some aspects, the support can be moved in two dimensions while the mass spectrum is being recorded.

This disclosure also provides for a method of nitrating indoles. In one aspect, the method of nitrating indoles includes cooling a mixture of iron porphyrin complex (e.g., (TPP)Fe^{III}) in a solvent (such as THF and dichloromethane), bubbling dioxygen gas through the mixture, adding an indole substrate to the mixture, and bubbling nitric oxide gas into the mixture. The method can further include separating and purifying the final nitrated products. In a separate aspect, the method of nitrating indoles follows the process described in U.S. Pat. No. 10,022,352 B2, which is incorporated herein by reference.

### Example 1

Since its initial development in the 1980s,¹ Matrix-Assisted Laser Desorption Ionization Mass Spectrometry (MALDI MS) has been applied to identify/detect a wide range of natural or synthetic compounds such as lipids,² peptides,³ proteins,⁴ glycans,⁵ oligonucleotides,⁶ and polymers.⁷ MALDI matrix molecules absorb photons and transfer energy to the analyte and assist with analyte desorption and ionization.⁸ Analytes are generally detected in positive ion mode as protonated, metal ion adducted, or radical cations, or in negative ion mode as deprotonated or radical anions.⁹ Over the past few decades, a large number of matrix molecules have been identified to expand applications,^{2, 10} and discovery of matrix molecules with broad applications for both positive and negative ions has been an ongoing quest, to simplify matrix choices during sample preparation and enhance target molecule detection sensitivity in complex mixtures. Recent research on dual-polarity MALDI matrix molecules has focused on lipid analysis, as lipids are a diverse family of compounds, with some ionizing better in positive ion mode and some in negative ion mode.² Several aromatic amine compounds have been reported as dual polarity matrices for lipid analysis, including 1,8-di(piperidinyl)-naphthalene (DPN), 11 nor-harmane,12 1,5-diaminonaphthalene (1,5-DAN),13, 14 3-aminophthalhydrazide (luminol),¹⁵ anthranilic derivative COOH—NHMe (IV),¹⁶ and hydralazine.¹⁷

Indole is an electron-rich compound widely distributed in biological systems such as proteins (amino acid tryptophan sidechain) and alkaloids.¹⁸ Some existing indole-related MALDI matrices are trans-3-indoleacrylic acid (IAA) for polymers and steroids,¹⁹⁻²¹ indole-3-pyruvic acid (IPA) for peptides and proteins,²² and 9H-pyrido[3,4-b]-indole (nor-harmane) for lipids, proteins, peptides, carbohydrates, and synthetic polymers.^{12, 23-26} Nitro-containing matrices generally have a nitro group attached to a benzene ring, such as 9-nitroanthracene (9-NA) for polymers and organic ligands,^{27, 28}2-nitrophloroglucinol (2-NPG) for protein multiple charging,²⁹ 5-nitrosalicylic acid (5-NSA) for glycan in source decay (ISD),³⁰ 3-hydroxy-4-nitrobenzoic acid (3H4NBA)/3-hydroxy-2-nitrobenzoic acid (3H2NBA) for peptide ISD^{31 32}, and 4-nitroaniline (PNA) for lipids.³³ Nitro substituted β-carboline (nor-harmane) and carbazole derivatives were also reported.^{34, 35}

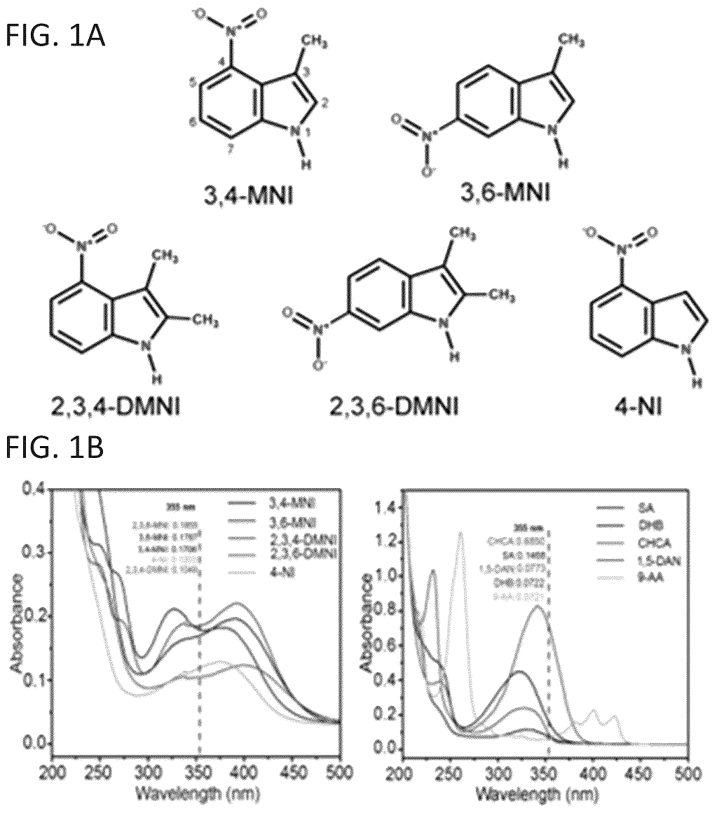

New MALDI matrix development is of continual research interest with the aim to further expand applications and improve analytical figures of merit. As matrix discoveries have often been by trials and errors, identifying a matrix design framework is especially helpful to speed up new matrix development. In this study, five indole derivatives 3-methyl-4-nitro-1H-indole (3,4-MNI), 3-methyl-6-nitro-1H-indole (3,6-MNI), 2,3-dimethyl-4-nitro-1H-indole (2,3,4-DMNI), 2,3-dimethyl-6-nitro-1H-indole (2,3,6-DMNI), and 4-nitro-1H-indole (4-NI) (FIG. 1) were synthesized and demonstrated for the first time to function as new dual polarity MALDI matrices with broad applications. Analysis with standard compounds and complex mixtures demonstrated that NI matrices effectively detect positive and/or negative ions of metabolites, lipids, peptides, proteins, glycans, oligonucleotides, polymers, and environmental pollutant polyfluoroalkyl substances. The wide-range matrix applicability helps simplify matrix choices during MALDI MS and imaging sample preparation and enhances detection sensitivity in complex mixture analysis. As slight indole ring substitution variations led to distinct matrix performance changes, nitro indole could function as a sensitive and versatile design platform for new matrix engineering.

### Experimental

### Materials

Chemicals for mass spectrometry analysis: peptide and protein calibration standards, 2, 5-dihydroxybenzoic acid (DHB), alpha-cyano-4-hydroxylcinnamic acid (CHCA), and sinapinic acid (SA) were obtained from Bruker Daltonics (Billerica, MA, USA). 1,5-DAN, 9-aminoacridine (9-AA), phosphoinositols (PI) Glycine max, fibrinopeptide B (fibB), ProteoMass Cytochrome c MALDI MS standard, beta-casein (b-CN), bovine serum albumin (BSA), Lacto-N-difucohexaose I (LNDFHI), perfluorooctanesulfonic acid (PFOS), and di-ammonium hydrogen citrate (dahc) were purchased from Sigma-Aldrich (St. Louis, MO, USA). Phosphocholine (PC) 16:0/16:0, phosphoethanolamine (PE) 16:0/18:1, and phosphatidate (PA) 16:0/18:1 were purchased from Avanti Polar Lipids (Alabaster, Al). Eggs, Bovine vitamin D whole milk, and frozen organic blueberries were purchased from a local grocery. All the other reagents, solvents, and salts were obtained from VWR International (Radnor, PA, USA).

### Synthesis and Characterization

3,4-MNI, 3,6-MNI, 2,3,4-DMNI, 2,3,6-DMNI, and 4-NI (FIG. 1) were synthesized following reference 36. ¹H Nuclear Magnetic Resonance Spectroscopy (NMR) was acquired on a Bruker DRX 400 MHz instrument, and high-resolution Electrospray Ionization (ESI) Mass Spectrum was acquired on a Waters Xevo G2-XS Qtof mass spectrometer. The UV-vis spectrum was recorded with a VWR UV-3100PC spectrophotometer.

### Sample Preparation

Matrix Performance with Standard Compounds:

The detection sensitivity test standards include PC 16:0/16:0, PE 16:0/18:1, and PA 16:0/18:1 at 10, 1, 0.1, 0.01 0.001 μM in methanol, PI Glycine max at 10, 1, 0.1, 0.01 0.001 ppm in methanol, fibB at 10, 1, 0.1, 0.01 0.001 μM in Milli-Q (MQ) water, BSA and b-CN at 10, 1, 0.1, 0.01 0.001 μM in 5 mM dahc ACN/water 1/1 and in MQ water, LNDFHI at 10, 1, 0.1, 0.01 0.001 ppm in MQ water, and PFOS at 10, 1, 0.1, 0.01 0.001 ppm in MQ and in tap water. 3,4-MNI, 3,6-MNI, 2,3,4-DMNI, 2,3,6-DMNI, 4-NI, CHCA, DHB, 9-AA, 1,5-DAN, and SA matrices were prepared at 5 mM in ACN/water 0.1% TFA for fibB, at 25 mM in ACN/water 0.1% TFA for BSA and b-CN analysis, and at 1 or 5 mM in methanol for other analysis. 1 μL of matrix was applied onto a Bruker Anchorchip target and dried, followed by 1 μL of standard solution applied on top. For BSA and b-CN protein analysis, samples and matrix were also mixed at 1.5:5 volume ratio, with 1 μL applied to the Anchorchip target. Sample spot homogeneity was evaluated with Scanning Imaging Microscopy (SEM, SU3500, Hitachi) and Epson Perfection V600 photo scanner.

### PFOS Quantitation in Water and Salt Tolerance:

Calibration and verification standards were prepared in MQ water (0, 0.05, 0.1, 0.5, 1, 2, 5, 10 ppb) and lab tap water (0, 0.05, 0.1, 0.5, 1, 2, 5, 10, 20, 50 ppb) with 10 ppb ¹³C8-PFOS Internal standard (IS) through isotopic dilution. Polypropylene centrifuge tubes and pipette tips were used to prevent background PFOS contamination. 1 μL of 3,4-MNI (1 mM in methanol) was applied to an Anchorchip target and dried. 3 μL of the standard solution was applied on top and dried. For salt tolerance study, 10 ppb PFOS solution was prepared in tap water with 0, 100 mM, 500 mM, and 1 M sodium chloride.

### Egg Lipids Analysis:

The lipids extraction followed the literature procedure¹¹: 1.0 mL methanol and 0.5 mL chloroform were added to 400 μL of homogenized egg (yellow and white) and sonicated for 5 minutes. After addition of 0.5 mL water, 0.5 mL chloroform, the mixture was sonicated again for 5 minutes and centrifuged for 10 minutes at 7000 rpm and the organic phase layer at the bottom was collected. 50 mM of DHB, 1,5-DAN, 9-AA, and NI matrices in methanol was mixed with lipid extract 2:1 v/v, with 1 μL applied to a Bruker MTP Anchorchip target.

### Milk Proteins Analysis:

Bovine vitamin D whole milk was diluted 1/100 in MQ water or 5 mM dahc ACN/water (1/1). 1 μL of the diluted milk was spotted on Anchorchip target and dried, with 1 μL matrix solution (25 mM in ACN/water 1/1, 0.1% TFA) applied on top, or the sample was mixed with matrix at 1:4 volume ratio. Tested matrix solutions include 3,4-MNI, SA, CHCA, DHB, and 1,5-DAN.

Blueberry extract and tissue imaging: One thawed frozen organic blueberry (˜2 g) was crushed and sonicated in 2 mL methanol for 30 minutes. The resulting methanol solution was centrifuged (10 min at 7000 rpm) and the supernatant was collected. 1 μL of 3,4-MNI, 3,6-MNI, 2,3,4-DMNI, 2,3,6-DMNI, 4-NI, 1,5-DAN, 9-AA, and DHB (1 or 5 mM in methanol, or 25 mM in ACN/water 0.1% TFA) was spotted onto Anchorchip target, and 1 μL of blueberry methanol extract was applied on top. For MALDI imaging, blueberries were stored at −20° C. and cut into 40 μm slices with a cryomicrotome (Tissue Tek II, Miles). The cut slices were thaw-mounted onto an ITO slide pre-sprayed with 10 mM 2,3,6-MNI in methanol (3×1 mL to cover the whole ITO slide) using a Testors Aztek airbrush. The resulting tissue-mounted slide was dried in a desiccator for 30 minutes and subsequently stored at −20° C. in a sealed container. Prior to the imaging experiment, 1 mL of 10 mM 2, 3,6-MNI in methanol was sprayed over the blueberry tissue area. For airbrush spray, the compressed air pressure was 20 psi and the distance between the slide and the sprayer tip was ˜15 cm.

### Matrix Vacuum Stability Measurements

The vacuum stability of 3,4-MNI, 3,6-MNI, 2,3,4-DMNI, 2,3,6-DMNI, and 4-NI matrices was measured in air using Quartz Crystal Microbalance (QCM). Briefly, 10 μL of 50 mM matrix solution in ACN/water (85/15) was applied onto the gold-coated surface of a quartz crystal electrode and dried. The crystals were secured between two stainless steel plates (top plate with cutouts, connected with screws) fitted to a MALDI imaging slide holder and admitted into the MALDI vacuum chamber for 4 hours. QCM oscillation frequencies were measured without matrix, with matrix deposition before and after 4-hr MALDI vacuum.

### MALDI MS/Imaging and ESI HRMS

The MALDI MS, collision-induced dissociation (CID, Argon collision gas 1.0 bar) MSMS, and imaging experiments were performed on a Bruker Daltonics rapifleX mass spectrometer equipped with a smart beam 3D laser (355 nm Nd:YAG, ≥100 μJ/pulse). MS mass calibrations below 4000 Da were performed with phosphorus red/CHCA matrix in positive and negative ion modes, and the 4000-100000 Da region positive ion mode calibration was performed with Cytochrome c and BSA protein standards mixed with SA matrix. BSA, b-CN, and milk protein spectra were acquired in linear mode, and all other experiments were acquired in reflectron mode. The acquired MS and MSMS spectra were analyzed with flexAnalysis 4.0.

Blueberry metabolites/lipids MALDI imaging experiments were acquired in reflectron positive and negative ion modes with 50 μm laser spot size, 51% absolute laser power in negative ion mode and 44% absolute laser power in positive ion mode, a raster width of 110 μm, number of scans of 2500, and laser frequency 5000 shots per second. The imaging data was acquired with Bruker flexControl 4.0 and flexImaging 5.0 software and analyzed with Bruker SCILS Lab (Version 2023a Core). The optical images of the ITO slides were scanned with an Epson Perfection V600 photo scanner. For blueberry metabolites identification, MALDI TOF/TOF CID MSMS experiments were performed on selected ions in extract and on tissue. ESI HRMS of the blueberry extract were performed on a Waters Xevo G2-XS Qtof instrument and analyzed with Masslynx 4.1 software. Metabolites accurate masses and MALDI MSMS spectra were searched against databases such as FOODB,³⁷ HMDB,³⁸ and LIPID MAPS.³⁹

### Results and Discussion

### NI Synthesis and Characterization

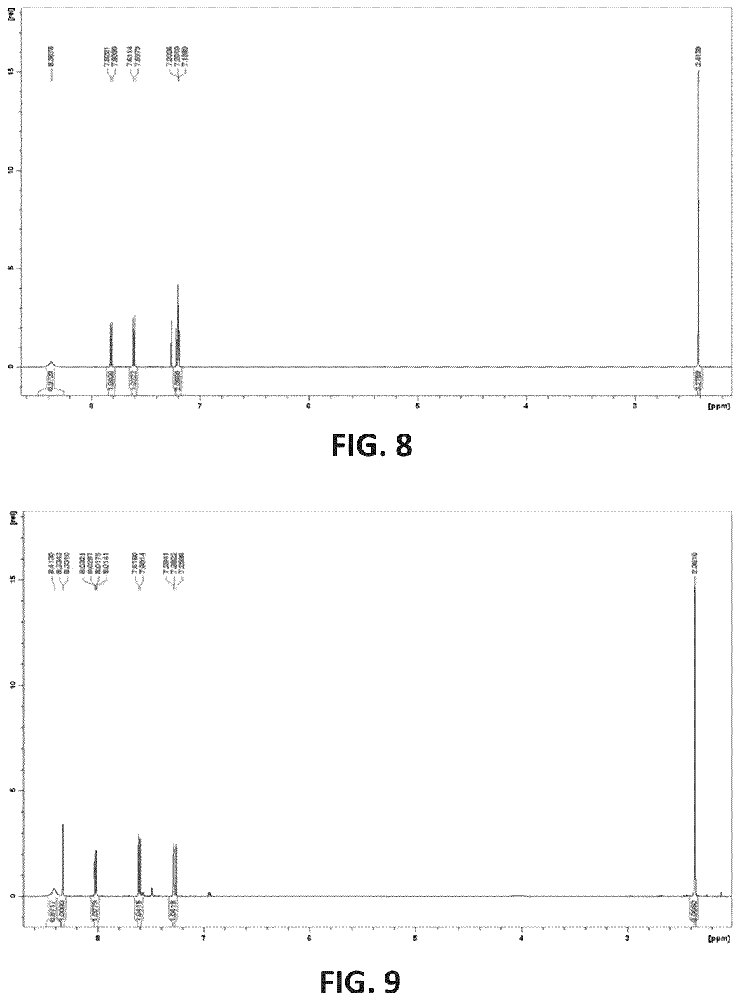

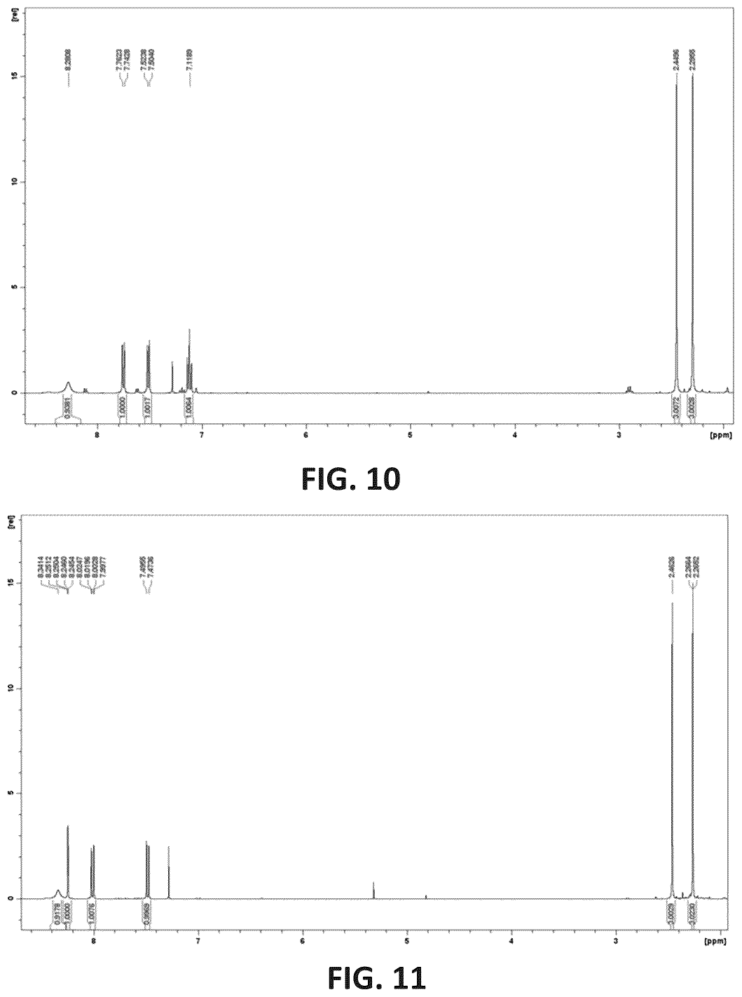

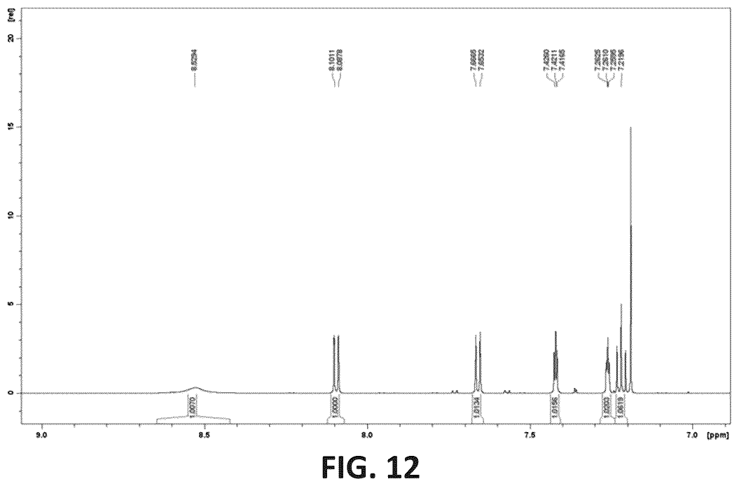

NI matrices were synthesized and purified according to the patented procedures for modulators of ATP-binding cassette transporters.³⁶ The compound identity was confirmed by ¹H NMR chemical shifts (in CDCl₃ at 298 K, FIGS. 8-12 and ESI HRMS: 3,4-MNI, [M+H]⁺ m/z=177.0662 (calc. 177.0664); 3-6-MNI: [M+H]⁺ m/z=177.0658 (calc. 177.0664); 2,3,4-DMNI: [M+H]⁺ m/z=191.0817 (calc. 191.0820); 2,3,6-DMNI, [M+H]⁺ m/z=191.0827 (calc. 191.0820); and 4-NI: [M+H]⁺ m/z=163.0501 (calc. 163.0508). All five NI matrices had efficient absorption at MALDI laser wavelength 355 nm, and the absorbance from high to low was A_{2,3,6-DMNI} 0.1855, A_{3,6-MNI} 0.1787, A_{3,4-MNI} 0.1706, A_{4-NI} 0.1203, A_{2,3,4-DMNI} 0.1049. The A_{2,3,6-DMNI}, A_{3,6-MNI}, and A_{3,4-MNI} values were higher than the absorbance of common matrices examined (A_{9-AA} 0.0721, A_{DHB} 0.0722, A_{1,5-DAN} 0.0773, A_{SA} 0.1468) except for CHCA (A_{CHCA} 0.6850) (FIG. 1).

### Detection Sensitivity, Homogeneity, and Ionization Mechanism

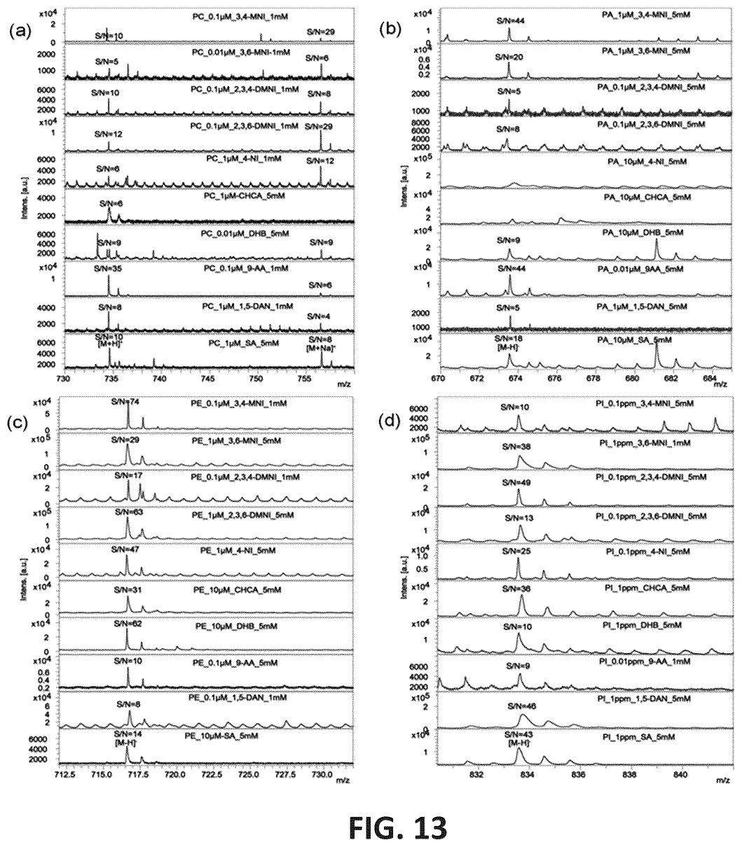

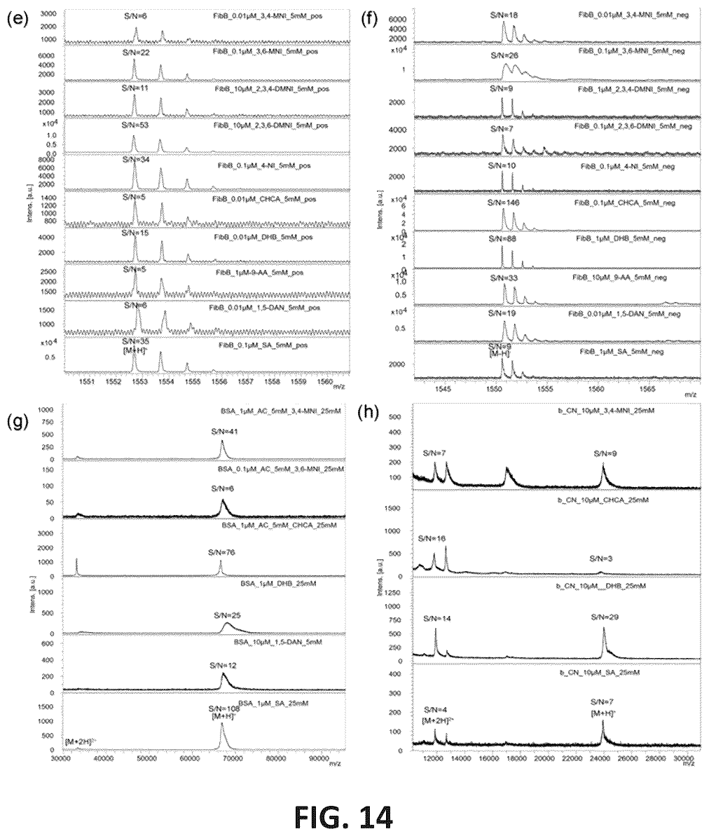

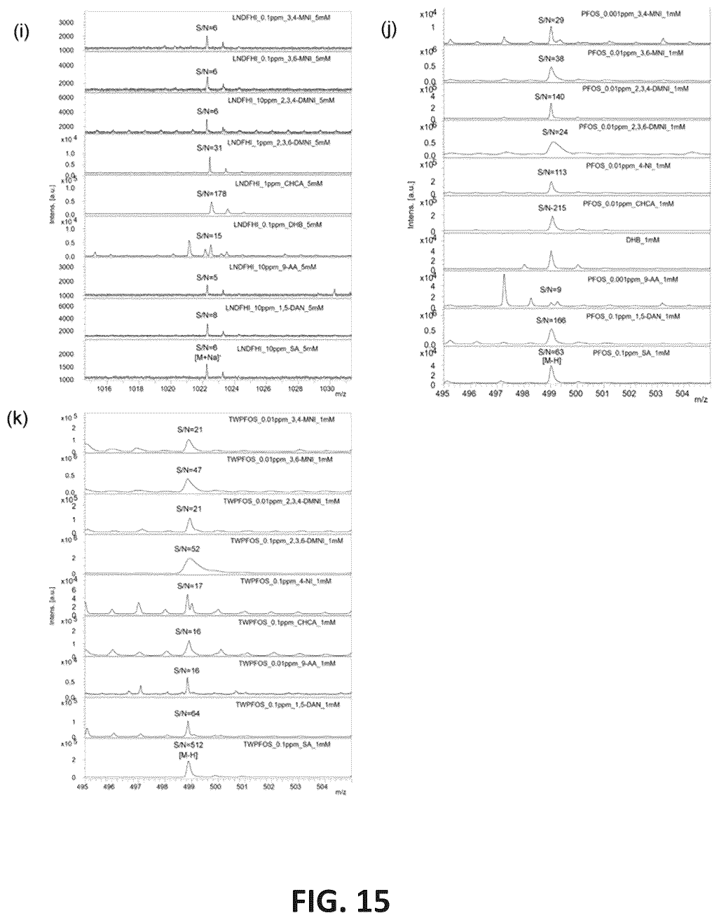

The detection sensitivity of nitro indole matrices 3,4-MNI, 3,6-MNI, 2,3,4-DMNI, 2,3,6-DMNI, 4-NI was compared with common matrices DHB, CHCA, SA, 1,5-DAN, and 9-AA for lipids (PC 16:0/16:0, PE 16:0/18:1, PA 16:0/18:1, PI Glycine max), peptide fibB, proteins (BSA and b-CN), glycan LNDFHI, and PFOS at concentration range 0.001-10 μM or ppm (Table 1, FIGS. 13-15). 1) Lipids: 3,6-MNI and DHB had the highest sensitivity for PC 16:0/16:0 [M+H]⁺ and [M+Na]⁺ cations (0.01 μM), 4-NI, CHCA, 1,5-DAN, SA had the lowest sensitivity (1 μM), and other matrices were intermediate (0.1 μM). 3,4-MNI, 2,3,4-DMNI, 9-AA, and 1,5-DAN were best for PE 16:0/18:1 [M−H]⁻ anions (0.1 μM), with CHCA, DHB, and SA least sensitive (10 μM). PA 16:0/18:1 [M−H]⁻ anions were detected at 0.01 μM with 9-AA, at 1 μM with 2,3-MNI, 2,6-MNI, 2,3,4-DMNI, 2,3,6-DMNI, 1,5-DAN, at 10 μM with DHB, SA, and was not detected by CHCA and 4-NI. PI Glycine max [M−H]⁻ anions were detected at 0.01 ppm with 9-AA, at 0.1 ppm with 2,3-MNI, 2,3,4-DMNI, 2,3,6-DMNI, 4-NI, and at 1 ppm with the others. 2) Peptide/protein/glycan analysis: 3.4-MNI, CHCA, and 1,5-DAN were the most sensitive for fibB peptide [M+H]⁺ cations and 3,4-MNI, 1,5-DAN most sensitive for [M−H]⁻ anions (0.01 μM). For BSA protein [M+H]⁺ and [M+2H]²⁺ ions, 3,6-MNI was the most sensitive (0.1 μM) and 3,4-MNI, CHCA, DHB, SA intermediate (1 μM). 3,4-MNI, CHCA, DHB and SA were effective for b-CN [M+H]⁺ and [M+2H]²⁺ ions (10 μM), and 3,4-MNI, 3,6-MNI, and DHB were better for LNDFHI glycan [M+Na]⁺ analysis (0.1 μM). 3) PFOS: in MQ water, 3,4-MNI and 9-AA gave the best sensitivity (0.001 ppm) for the [M−H]⁻ anions, other NI matrices showed similar performance as CHCA and SA (0.01 ppm), while DHB had matrix ion interference. In complex solutions such as tap water, 3,4-MNI, 3,6-MNI, 2,3,4-DMNI and 9-AA maintained good sensitivity (0.01 ppm) while CHCA and SA sensitivity dropped to 0.1 ppm. In summary, 3,4-MNI demonstrated the best overall sensitivity for all compounds examined, while the other NI matrices showed high sensitivity for various compounds. Table 1 comparison also correlated the distinct performance shifts of NI matrices with indole ring substitution variations, such as with/without methyl groups and the position of the methyl and nitro groups, indicating that nitro indole framework could function as a sensitive and versatile platform for new matrix designs.

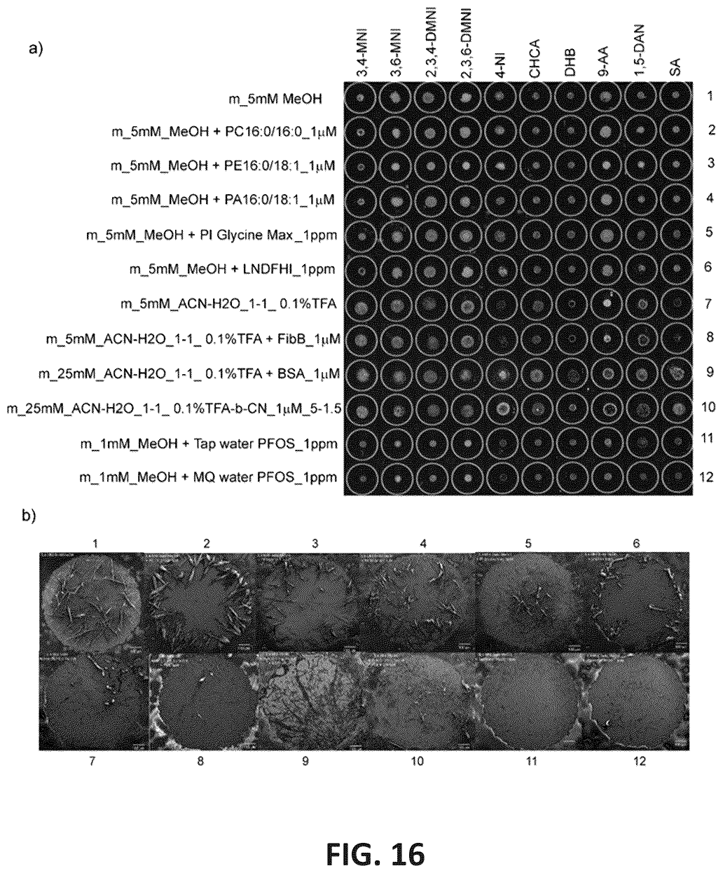

SEM and photo scanned images (FIG. 16) of sample spots on Anchorchip target demonstrated that 3,4-MNI, 3,6-MNI, 2,3,6-DMNI, and CHCA had more homogeneous and reproducible crystalline formation compared to 2,3,4-DMNI, 4-NI, DHB, 9-AA, 1,5-DAN, and SA. The 3,4-MNI and 3,6-MNI crystalline patterns changed dramatically based on solvent and sample choices, indicating the efficient and versatile matrix-sample interactions, correlating with their detection sensitivity for a wide variety of compounds.

The effectiveness of NI MALDI matrices is related to the structural combination of the electron-rich indole ring with C4/C6 nitro and C2/C3 methyl substituents. The effect of methyl and nitro substituents on indole proton affinity (PA) has been reported.^{51 52} Among the unsubstituted indole ring positions (N1, C2-C7, FIG. 1), C3 site has the highest proton affinity (922.5 KJ/mol) and N1 has the lowest (866.6 KJ/mol), which explains the well-known protonation preference of indole at C3 site.⁵¹ Methyl substituent releases electron density and increases PA for all positions except for the ipso one. Nitro substituent withdraws electrons and decreases PA values for all indole ring positions.⁵¹ The sum effect is multiple sites available for protonation and deprotonation, which may contribute to the superb dual polarity ionization efficiency of the NI matrices.

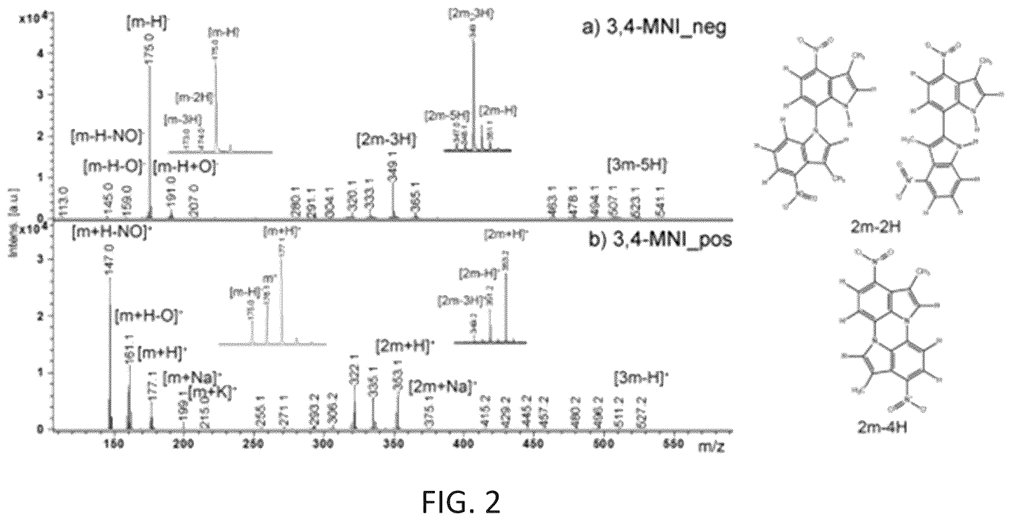

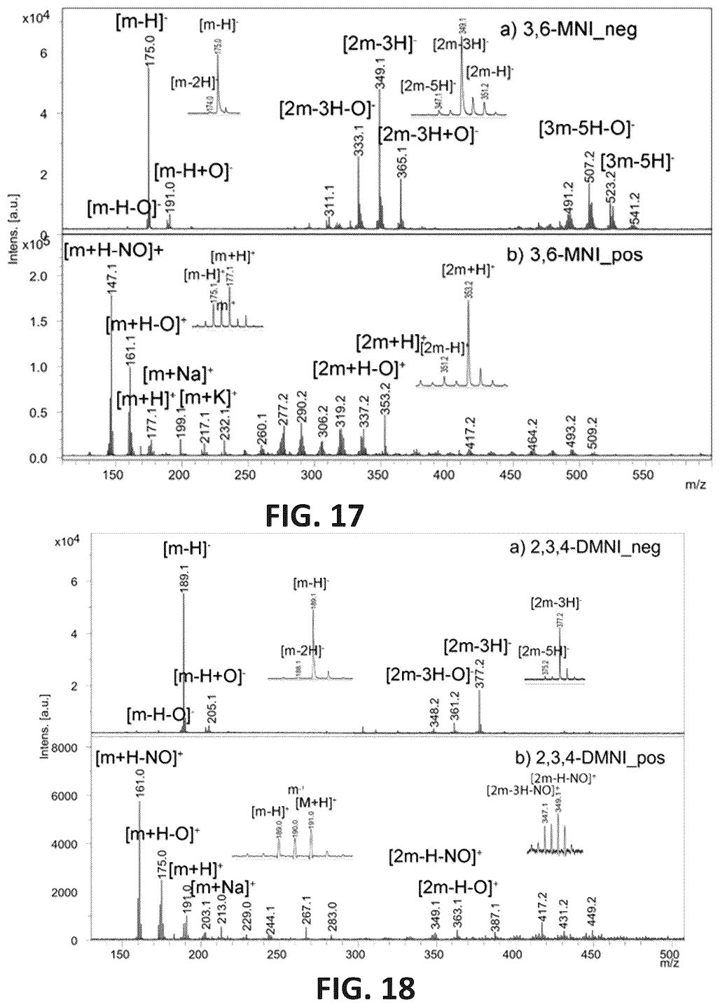

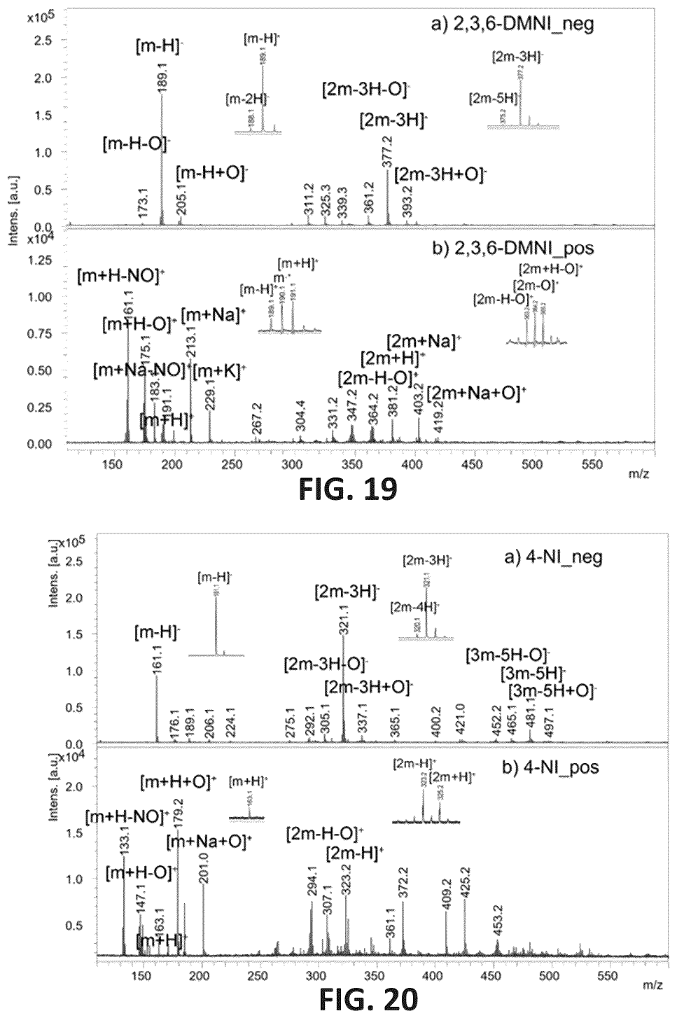

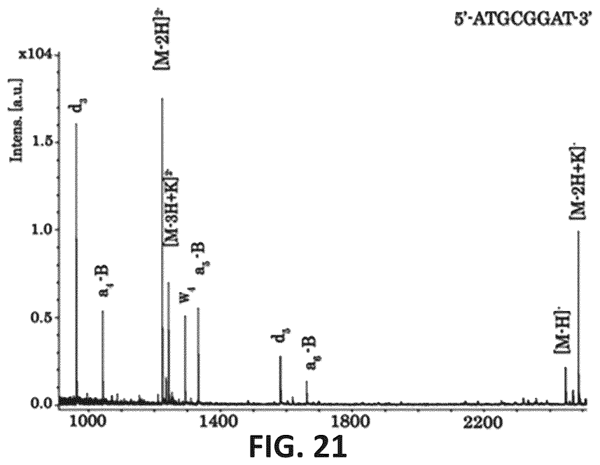

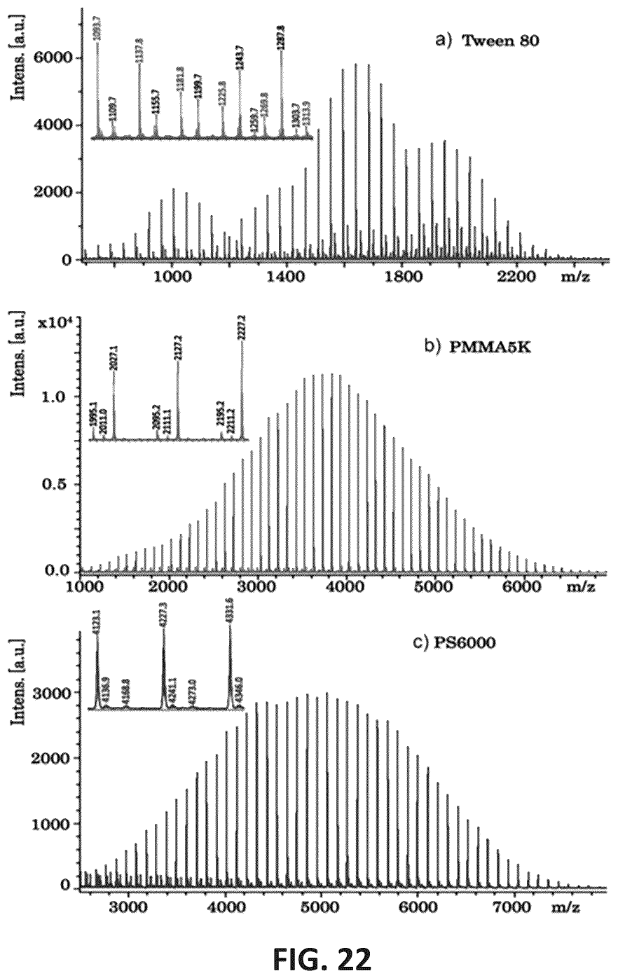

NI matrix background ions observed included monomeric or multimeric matrix cations and anions, such as [m+H]⁺, m⁺, [m−H]⁺, [m+Na]⁺, [m+K]⁺, [2m+H]⁺, [2m−H]⁺, [3m−H]⁺ and [m−H]⁻, [m−2H]⁻, [m−3H]⁻, [2m−H]⁻, [2m−3H]⁻, [2m−5H]⁻, [3m−5H]⁻. The loss/gain of O and loss of NO from [m−H]⁻, [2m−3H]⁻, and [3m−5H]⁻ are related to the aromatic nitro group (FIG. 2 and FIGS. 17-20).⁴⁰ The [m−H]⁺ ions are not commonly observed in MALDI ionization, but have been reported for some secondary and tertiary amines.^{27, 41, 42} The proposed mechanisms include 1) matrix protonation [m+H]⁺ followed by loss of H₂, 2) hydrogen atom transfer from m⁺, and 3) hydride abstraction from neutral matrix molecules.⁴¹ Considering the conjugated indole ring structure of NI, mechanism 1) seems less likely, while mechanisms 2) and 3) are possible. Dimeric cations [2m−H]⁺ could be non-covalent attachment of m with [m−H]⁺ or protonation of the covalently linked dimer with 2H loss, [2m−2H+H]⁺. Anions [2m−3H]⁻, [2m−5H]⁻, [3m−5H]⁻ could be hydrogen loss from the covalently bonded dimers or trimers as [2m−2H−H]⁻, [2m−4H−H]⁻, and [3m−4H−H]⁻. Solvent choices did not affect the ion species identified in the NI matrix MS spectra, but the ACN/H₂O combination induced higher multimeric matrix ions than methanol solvent. Matrix radical ions were minor compared to [m+H]⁺/[m+H−NO]⁺ or [m−H]⁻, and analyte ionization may mainly follow the matrix analyte proton transfer mechanism.^{8, 9} The presence of [m+Na]⁺ and [m+K]⁺ ions suggested possible matrix analyte cation transfer. Observed analyte ions for standards tested in Table 2 included [M−H]⁻, [M+H]⁺, [M+2H]²⁺, and [M+Na]⁺. Additionally, NI matrices were tested effective for short chain oligonucleotides anions ([M−H]⁻, [M−2H+K]⁻, [M−2H]²⁻, [M−3H+K]²⁻) and polymer [M+Na]⁺ and [M+Ag]⁺ cations (example spectra with 3,4-MNI matrix provided in FIG. 21, 22). For proteins, ammonium salts enhanced analyte protonation by reducing analyte sodium ion binding.

### Vacuum Stability Measurements

MALDI matrix vacuum stability information is useful for MALDI MS and imaging experiments that take long hours on high vacuum instruments.^{15, 43} The weight loss of matrix-coated MALDI imaging ITO slides after placement in MALDI chamber for a period of time can be measured with a semi-micro analytical balance.⁴³ QCM is a sensitive mass balance capable of measurement of nanogram to microgram mass changes. The piezoelectric thin quartz disk plated by gold electrodes oscillates at a defined frequency under electric potential and undergoes frequency change with addition or removal of mass (˜20 ng/Hz).⁴⁴ QCM has been applied in MALDI to determine neutral particle yield and matrix desorption as a function of elevated temperature. 45, 46

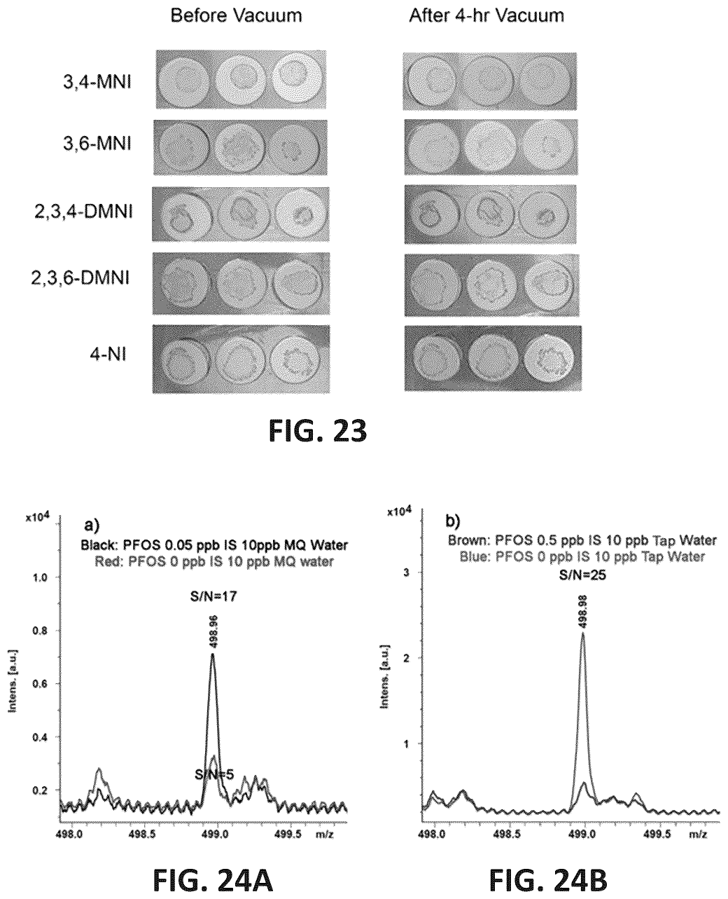

In this study, NI matrices were spotted on gold-plated quartz crystal electrodes and the oscillation frequencies were measured without matrix (f0), with matrix deposition before vacuum (f1), and with matrix deposition after 4-hr vacuum (f2). The deposited matrix masses (a sum of matrix and residual solvents) were calculated by (f0−f1) Hz×20 ng/Hz. The mass loss after 4-hour vacuum was calculated by (f1−f2) Hz×20 ng/Hz, and the percentage of loss was calculated by (f2−f1)/(f0−f1). The results (Table S1) showed that at 2.0e-7 Torr after 4 hours, 3,4-MNI and 3,6-MNI were the least stable with 76.5% and 77.8% mass loss respectively, 4-NI was most stable (29.5% loss), while 2,3,4-MNI (33.7% loss) and 2,3,6-DMNI (53.2%) had an intermediate mass loss. RSD % of three measurements seemed to correlate with the matrix crystallization homogeneity on gold (Table S-1, FIG. 23), with RSD less than 5% for more homogeneous 3,4-MNI and 3,6-MNI, and ˜50% for 2,3,4-DMNI which had the worst coffee ring effect during dried-droplet crystallization. This observation can be explained by that the vacuum sublimation rate is affected by the total matrix crystalline surface areas, and that nonhomogeneous crystallization leads to bigger surface area variations. Overall, the QCM matrix dried droplet method provided a sensitive and low matrix consumption method for MALDI matrix vacuum stability evaluation.

### Complex Mixtures Analysis

### Direct PFOS Quantitation in Tap Water

PFOS is one of the primary per- and poly-fluoroalkyl substances (PFAS) that are linked to harmful health effects and persist in the environments (water, soil, food chains).^{47, 48} PFOS quantitative analysis in complex mixtures is routinely performed with liquid chromatography (LC)-MS/MS. With multiple reaction monitoring (MRM) on tandem quadrupole instruments the typical instrument limit of quantitation (LOQ) is 0.5 ppb.^{49, 50} Lower LOQ can be achieved with high resolution (HR) MS precursor ion accurate mass, e.g. 0.05 ppb with Qtof MS^{E 51} and 0.025 ppb (method LOQ 0.1 ppt multiplied by SPE enrichment factor 250x) with orbitrap parallel reaction monitoring (PRM).⁵² Combined with solid phase extraction (SPE), the methods LOQ could reach ppt and sub ppt levels for both MRM and HRMS meathods.⁵²⁻⁵⁴

Compared to the LC-MS/MS platform, MALDI MS and imaging is an emerging technique that potentially allows direct PFAS analysis in complex mixtures,⁵⁵⁻⁵⁸ with additional benefits of high throughput, minimal solvent consumption, and spatial distribution information on tissues. The challenge is finding the optimum matrix substances that are complex mixture tolerant with good sensitivity/reproducibility. PFOS MALDI MS quantitation with 1,8-bis(tetramethylguanidino)-naphthalene⁵⁷ and desorption/ionization on porous silicon (DIOS)⁵⁸ as matrices were reported for SPE-treated tap water, but direct PFOS analysis in tap water with MALDI has not been reported to our knowledge.

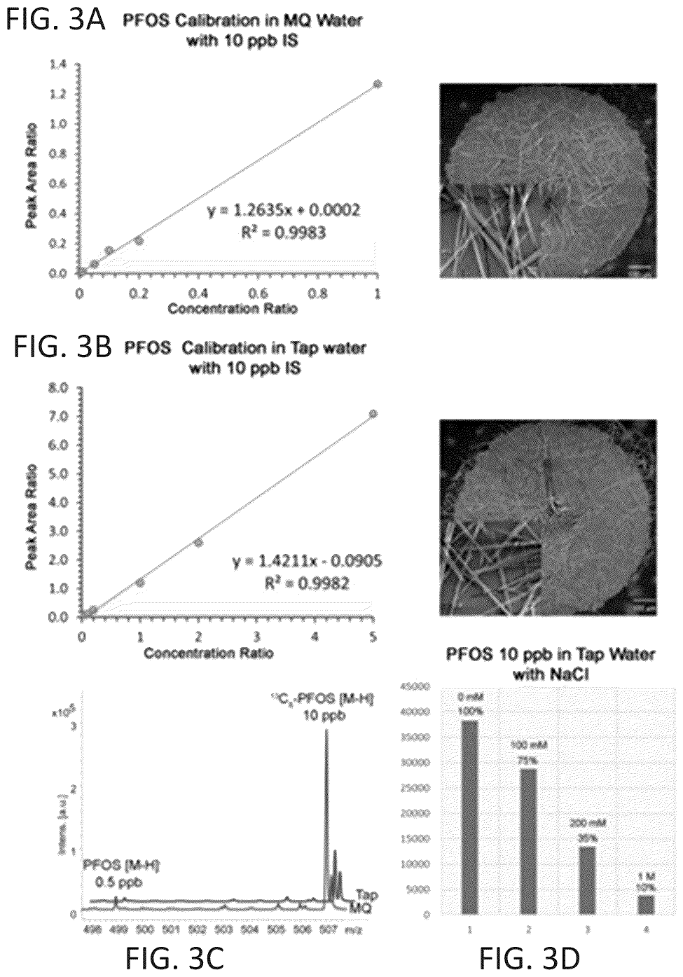

FIGS. 3a and 3b compared the 3,4-MNI MALDI PFOS calibration curves and SEM images in MQ water versus in tap water. In MQ water, six PFOS concentration levels (0.05, 0.1, 0.5, 1, 2, 10 ppb) with 10 ppb ¹³Cs-PFOS IS were spotted onto an Anchorchip target in six replicates. Peak area ratios of PFOS [M−H]⁻ m/z 498.99 and ¹³C8-PFOS [M−H]⁻ m/z 507.02 were plotted versus their concentration ratios. For the linear range of 0.05-10 ppb, the regression coefficients were at 0.9983, the average relative standard deviation was 5%, and the verification concentration of 5 ppb had a calculated value of 4.84 ppb (3.2% error) and RSD of 4% (Table 13). In tap water, six PFOS concentration levels (0.5, 1, 2, 10, 20, 50 ppb) with 10 ppb ¹³C8-PFOS IS were spotted onto an Anchorchip target in six replicates. For the linear range of 0.5-50 ppb, the regression coefficients were at 0.9982, the average relative standard deviation was 4%, and the verification concentration of 5 ppb had a calculated value of 5.25 ppb (5.0% error) and RSD of 3% (Table S2-2). SEM images (FIG. 3a, 3b) revealed that 3,4-MNI formed microscopic needle shaped crystals over the hydrophilic anchor areas on the Anchorchip target. This crystallization pattern was reproducible from spots to spots in MQ water or tap water, demonstrating efficient sample matrix interaction and thus robust quantitative measurements.

The PFOS LOQ with 3,4-MNI in MQ water (0.05 ppb) was superior to that in tap water (0.5 ppb) due to ion suppression in tap water. FIG. 3c showed the ion intensity difference for PFOS 0.5 ppb with 10 ppb IS in tap water and in MQ water. FIGS. 24A and 24B compared the LOQ and zero concentration spectra. Note that the MQ water spectra were acquired at lower laser power to ensure adequate peak resolution so that both PFOS and IS ions were clearly resolved from the background noise peaks, which was critical for accurate peak area integration. Salt tolerance of the 3,4-MNI matrix for PFOS analysis was evaluated by the optimum spectra comparison of 10 ppb PFOS in tap water with 0, 100 mM, 200 mM, and 1 M NaCl (FIG. 3d). The peak area of m/z 498.99 [M−H]⁻ declined to 75% (100 mM), 35% (500 mM), and 10% (1 M) compared with no NaCl added (FIG. 3b), suggesting good salt tolerance of 3,4-MNI at medium NaCl level (100 mM).

To summarize, direct MALDI MS analysis of PFOS in tap water with 3,4-MNI matrix demonstrated LOQ, precision, and accuracy comparable to standard LC-MS/MS MRM methods, with the additional benefits of complex mixture/salts tolerant, fast (acquisition time in seconds versus minutes per sample with LC-MS/MS), and environmentally friendly (minimal solvent consumption), applicable for ppb level environmental contamination and environmental engineering PFOS analysis. PFOS analysis in MQ water had the LOQ (0.05 ppb) comparable to LC-MS/MS HRMS methods, which suggests when coupled to SPE enrichment this method is applicable to sub-ppt level detection in drinking water. In addition, this method is suitable for automation and high throughput as the matrix was pre-spotted on a 384-spot target before sample spotting, and automatic target movement (smart complete) was applied during signal averaging.

### Milk Protein Analysis

3,4-MNI demonstrated equivalent performance as CHCA, DHB, and SA matrices for protein standards analysis (Table 1). To further evaluate 3,4-MNI performance for proteins in complex mixtures bovine vitamin D whole milk was chosen as a test sample. Milk is an important nutritional source of lipids, proteins, amino acids, vitamins, and minerals.⁵⁹ Proteins make up on average 3.5% of bovine milk, with 80% caseins and 18% whey proteins. Caseins are a family of related phosphoproteins with five types (aS1-CN, aS2-CN, b-CN, k-CN, g-CN) and over 50 naturally occurring variants. Major whey proteins include beta-lactoglobin (bLG), alpha-lactalbumin (aLA), BSA, and immunoglobulins (Igs)⁶⁰.

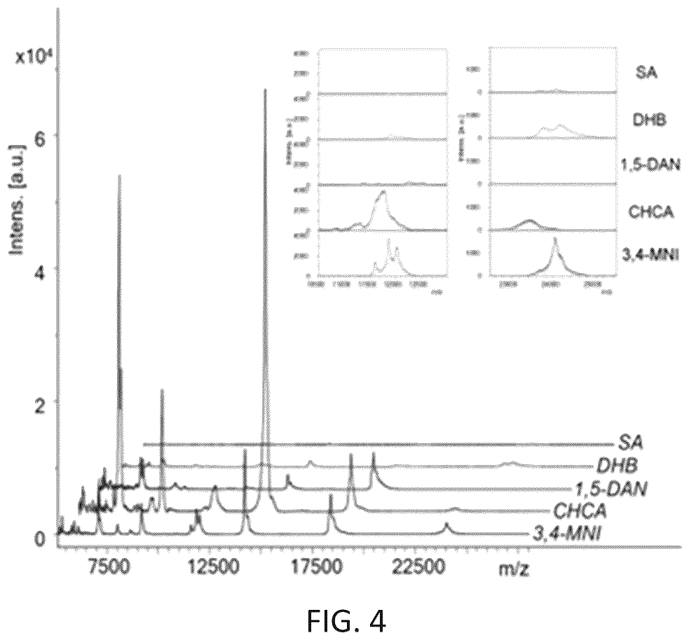

The whole milk sample was simply diluted 1/100 with MQ water or 5 mM dahc ACN/water 1/1 without the fat and cell debris removal centrifugation step.⁶¹ FIG. 4 compared the MS spectra acquired with 3,4-MNI, CHCA, 1,5-DAN, DHB and SA matrices (25 mM in 50/50 ACN/water 0.1% TFA) in positive ion mode. The protein ions observed were tentatively assigned based on reported milk protein molecular weights⁶⁰: 24 KDa/12 KDa (singly/doubly charged) as b-CN, 23.6 KDa/11.8 KDa as aS1-CN, 18 KDa/9 KDa as bLG, and 14 KDa/7 KDa as aLA. In FIG. 4 and insert, 1,5-DAN and CHCA failed to detect the singly charged b-CN ions (24 KDa) and the doubly charged ions were weak; SA and DHB produced singly and doubly charged b-CN ions but at weaker ion intensity compared to the 3,4-MNI matrix. 3,4-MNI showed a lower intensity of the singly charged aS1-CN ions but a higher intensity of the doubly charged ions compared to DHB and SA. CHCA was the most sensitive for aLA at 14 Kda/7 Kda. For optimum detection sensitivity of all identified protein ions, 3,4-MNI was the best choice, least affected by ion suppression by complex milk constituents.

### Egg Lipids Analysis

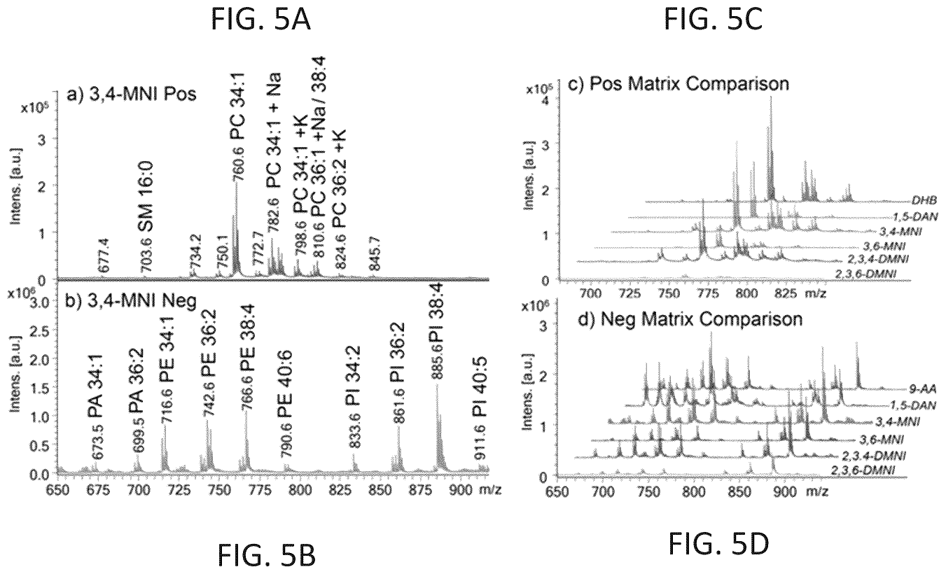

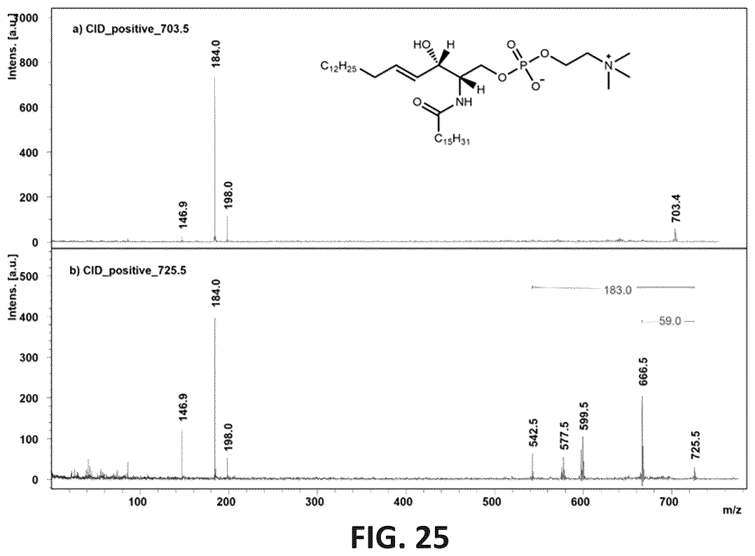

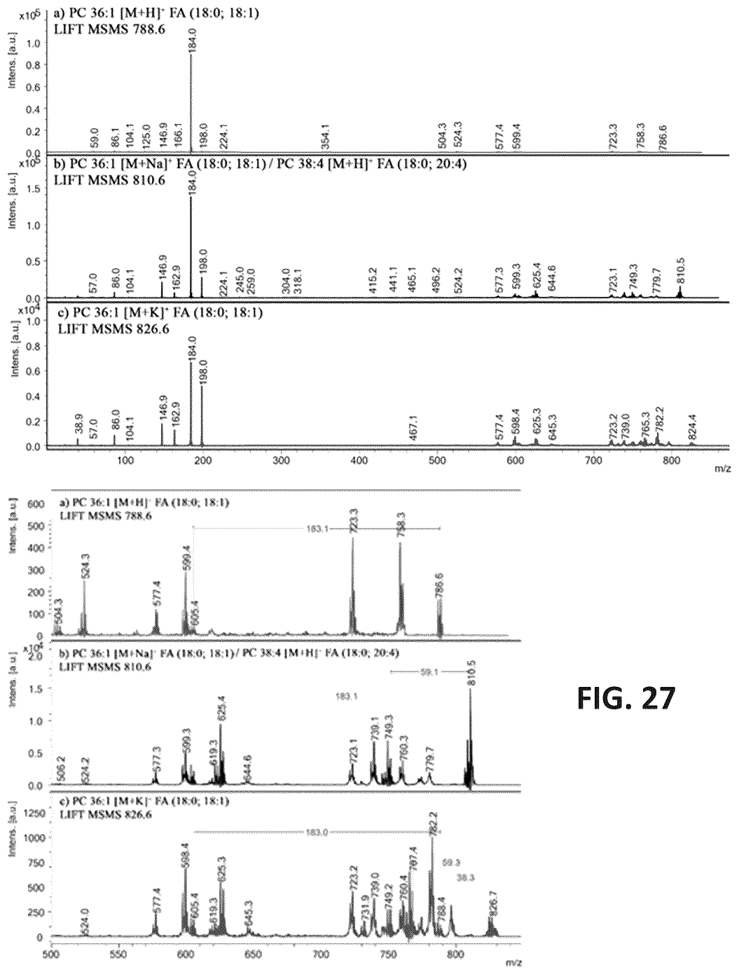

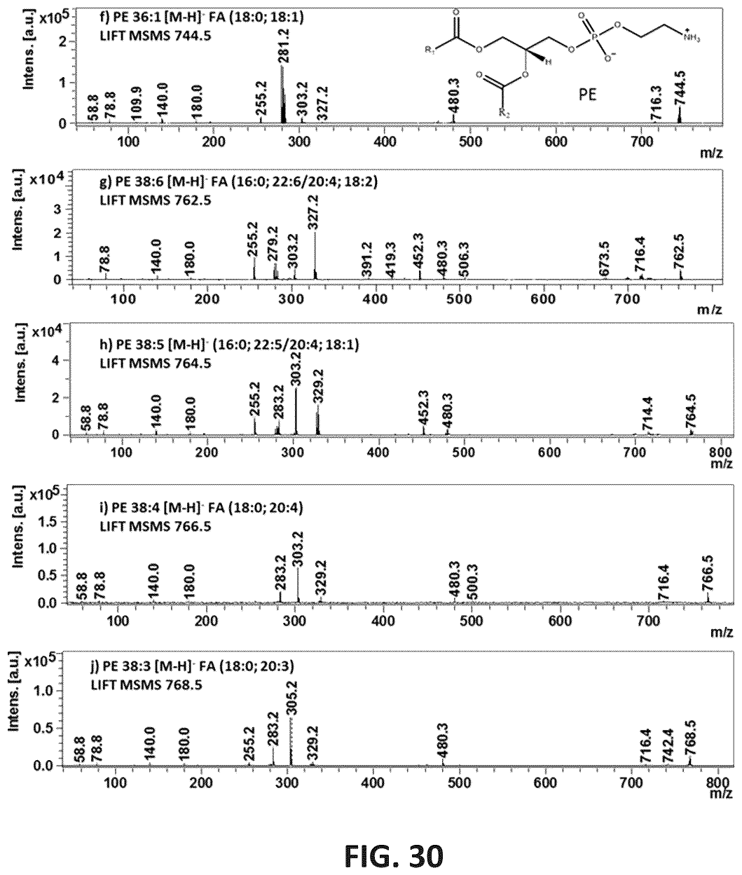

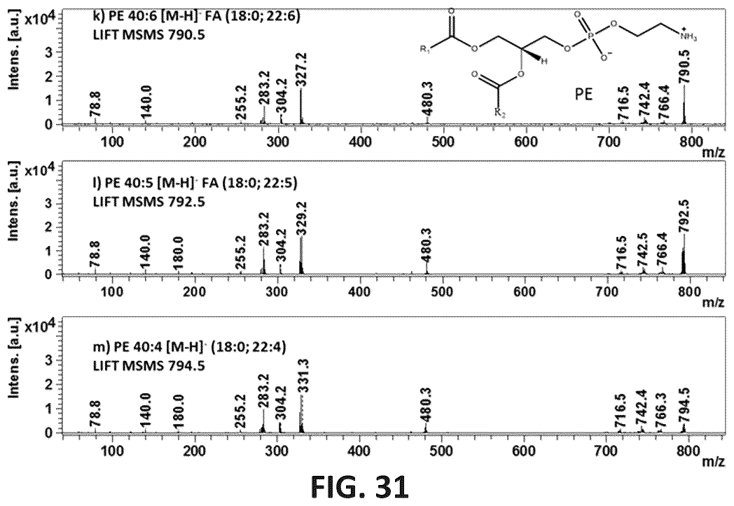

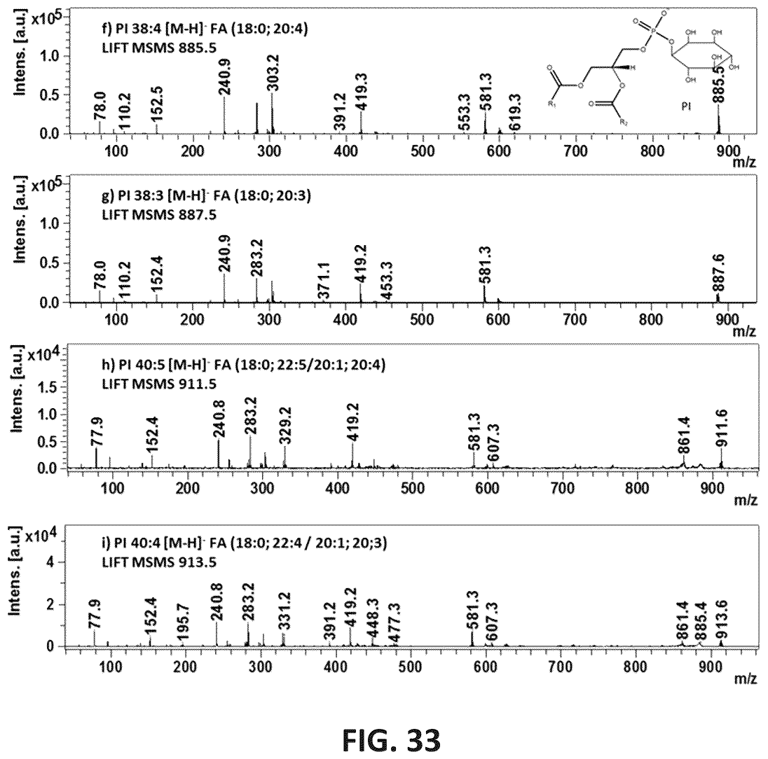

For lipids mixture analysis, egg lipids extract was selected to evaluate the performance of 3,4-MNI, 3,6-MNI, 2,3,4-DMNI, and 2,3,6-DMNI in comparison with common lipid MALDI matrices such as 9-AA for negative ions,⁶² DHB for positive ions,⁶³ and 1,5-DAN for positive and negative dual-polarity ions.¹³ (4-NI was not included due to its lower detection sensitivity for lipids in Table 1). MS spectra were acquired at optimum laser power for each matrix. The observed ions were assigned based on reference reported assignments^{11, 64, 65} and the MALDI TOF/TOF CID MSMS spectra (Table S3, FIGS. 25-33) in reference to general lipid MSMS fragmentation patterns.⁶⁶⁻⁷⁰ FIG. 5a and FIG. 5b illustrated the assigned phospholipid ions in positive and negative ion modes with 3,4-MNI matrix. The detected lipid species included sphingomyelin (SM), PC, PE, PA, and PI.

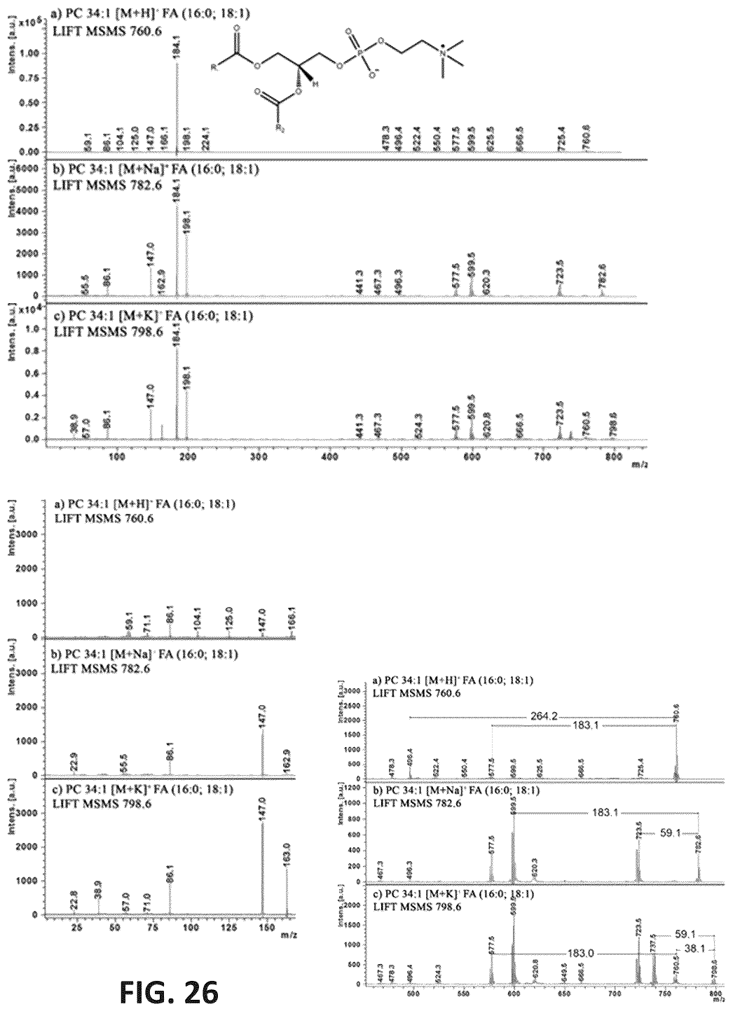

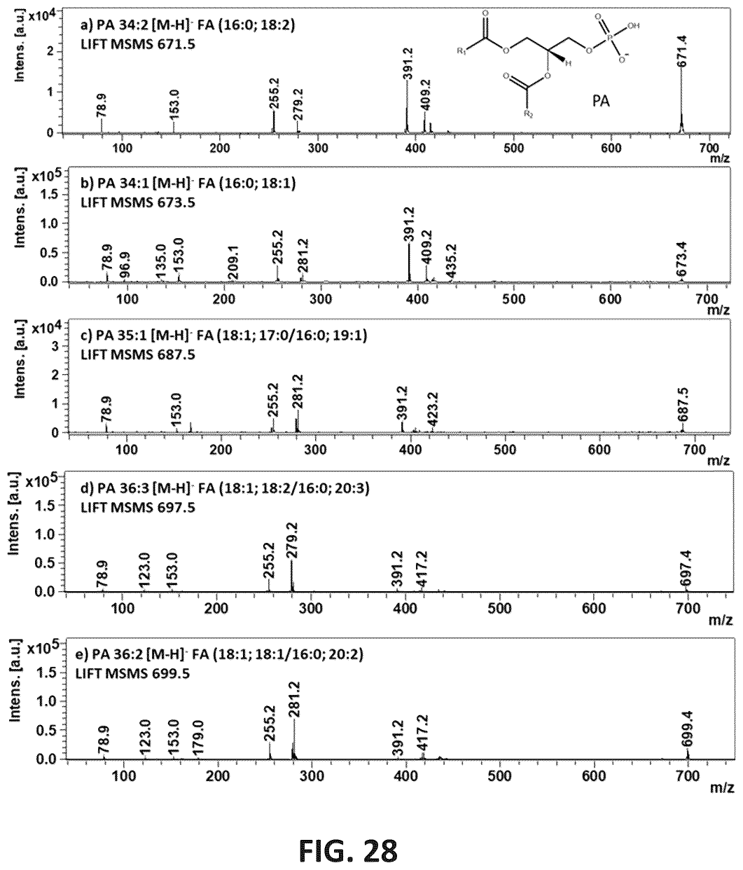

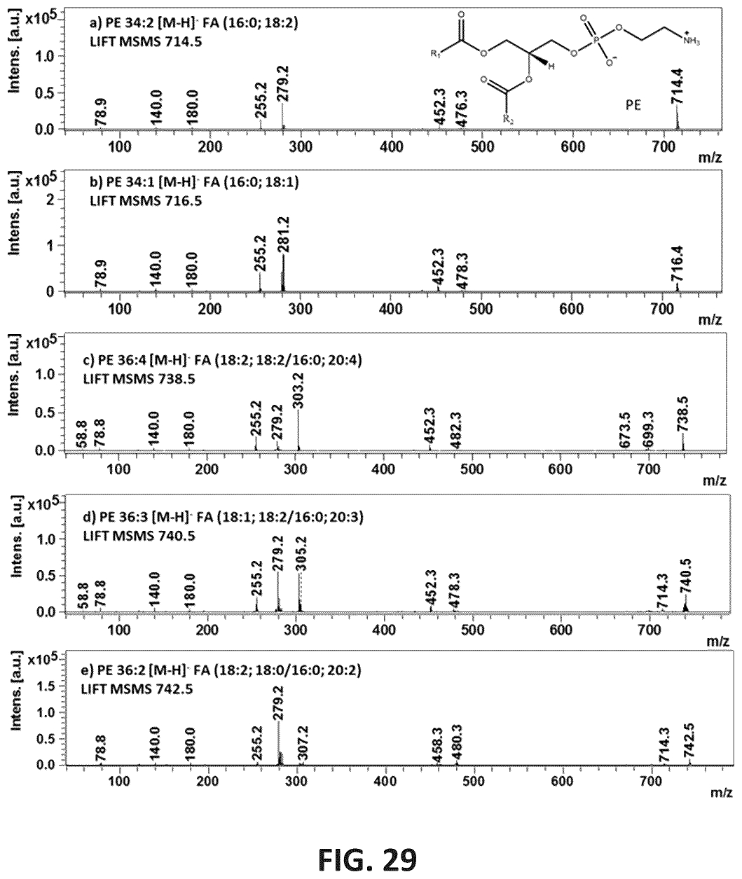

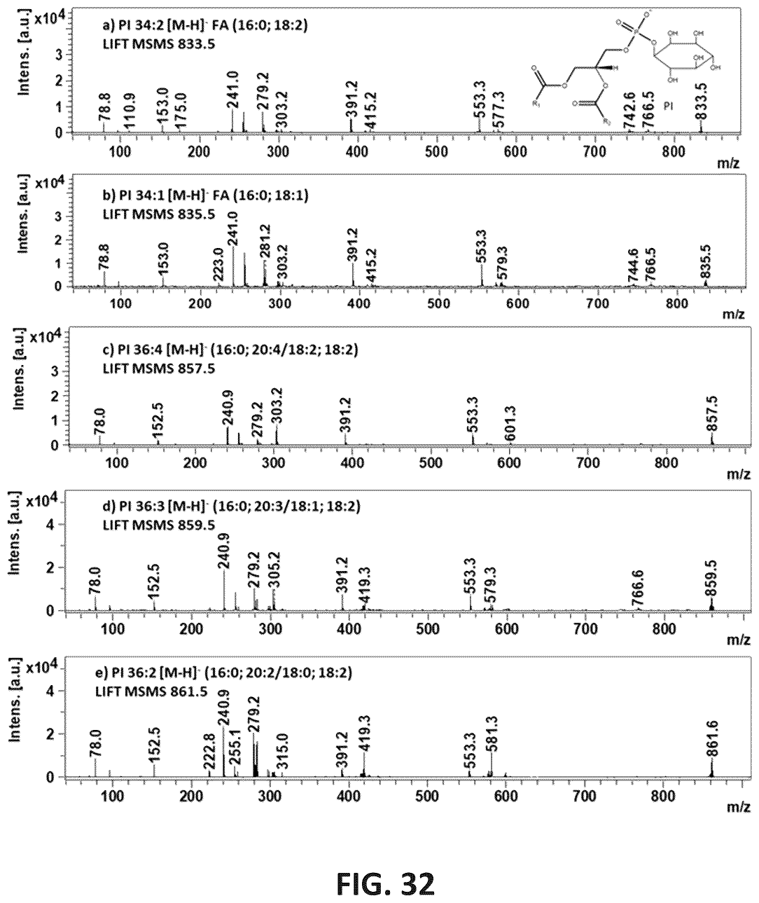

In positive ion mode, SM 16:0 [M+H]⁺, [M+Na]⁺ and PC 34:1, 34:2, 36:1, 36:2 [M+H]⁺, [M+Na]⁺, [M+K]⁺ ions were identified (FIG. 5a, Table S3, FIGS. 25 and 26). For example, FIG. 26 compared the MALDI TOF/TOF CID spectra for m/z 760.5, 782.5, and 798.5. Ions of m/z 760.5 were assigned as [M+H]⁺ of PC 34:1 with major fragments m/z 184.1 (PC head group, C₅H₁₄PO₄NH), 478.4 [M+H-C₁₈H₃₄O₂]⁺, 496.4 [M+H-C₁₈H₃₂O]₊, 504.4 [M+H-C₁₆H₃₂O₂]⁺, 522.4 [M+H-C₁₆H₃₀O]⁺, and 577.5 [M+H-C₅H₁₄PO₄N]⁺. Ions m/z 782.5 and m/z 798.5 were assigned as [M+Na]⁺ and [M+K]Y of PC 34:1, respectively. The loss of Sn2 fatty acyl is sterically more favorable than Sn1 fatty acyl,⁶⁸ which could correlate to the higher intensity of m/z 496.4, however, Sn1 and Sn2 positional isomer ratios cannot be clarified with MALDI MSMS spectra only as the ion abundance ratio is dependent on the headgroup, fatty acyl identity, and instrument conditions.^{71, 72} Therefore, sn1 and sn2 positions were not specified in the assignments. In negative ion mode, PA, PE, PI ions were identified (FIG. 5b, Table S3, FIGS. 28 to 33). In the MSMS spectra of m/z 671-699 (FIG. 28), m/z 79 and 153 were PA head group fragment ions. In the MSMS spectra of m/z 714-794 (FIG. 29 to 31), m/z 122 and m/z 140 corresponded to the PE ethanolamine phosphate ions with and without water loss. PI head group ions m/z 223, 241, 259, 297, 315 were present in the MSMS spectra for m/z 833-913 (FIG. 32, 33).

Egg lipids MALDI MS spectra matrix comparison (FIG. 5c, 5d) demonstrated that 3,4-MNI, 3,6-MNI, 2,3,4-DMNI, and 2,3,6-DMNI all functioned as effective dual polarity ion matrices, and 3,4-MNI had the best overall performance for positive and negative ions. In positive mode, the ion intensity of PC 34:1 in egg lipids extract followed the order DHB-3,4-MNI>1,5-DAN-2,3,4-DMNI>3,6-MNI>2,3,6-DMNI. In negative ion mode, the ion intensity orders were 3,4-MNI>2,3,4-DMNI>9-AA-3,6-MNI>1,5-DAN-2,3,6-DMNI (PI34:2), 3,4-MNI>2,3,4-DMNI>9-AA-1,5-DAN>3,6-MNI>2,3,6-DMNI (PE 38:4), 1,5-DAN>9-AA-3,4-MNI-2,3,4-DMNI>3,6-MNI>2,3,4-DMNI (PA 34:1).

### Blueberry Metabolites/Lipids Tissue Imaging

MALDI imaging is an emerging tool to visualize the distribution of a wide variety of plant metabolites across organs and tissues,^{73, 74} which is intrinsically challenging due to chemical complexity and potential ion suppression on tissues. DHB matrix has been applied to positive ion plant metabolites imaging, e. g. blueberry,⁷⁵ strawberry,^{76, 77} tomato,⁷⁸ and Ginkgo biloba.⁷⁹ In negative ion mode, 9-AA for tomato,⁷⁸ 1,5-DAN for strawberry,⁸⁰ 1,8-bisdimethyl-amino naphthalene (DMAN) for Medicago truncatula,⁷³ 2,4,6-trihydroxyacetophenone (THAP) for blueberry,⁸¹ Michler's ethylketone for Chinese-yew seed,⁸² and LDI (no matrix) for Ginkgo biloba⁷⁹ were reported. For blueberry metabolite imaging, reported studies were mostly limited to anthocyanins and polyphenols with DHB⁷⁵ or THAP⁸¹ as a matrix.

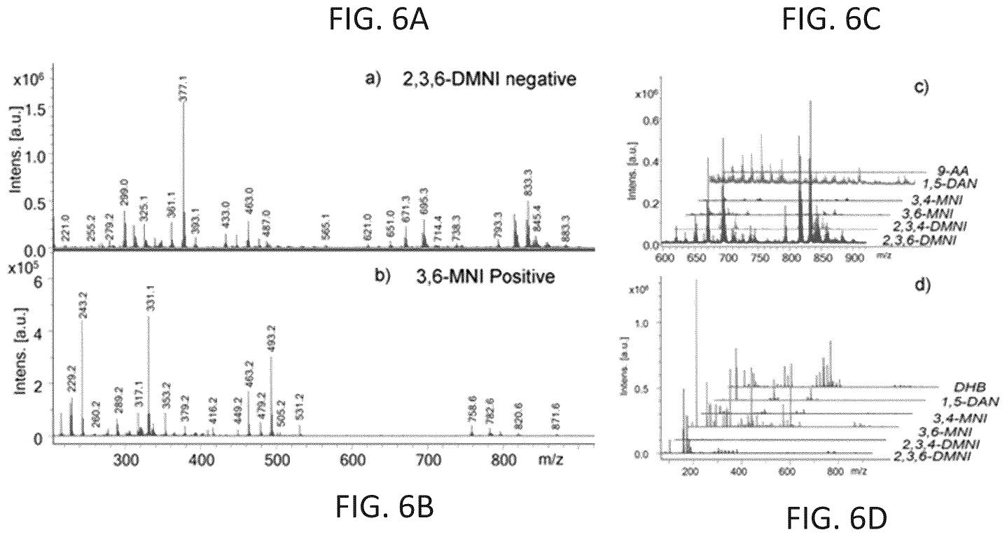

In this study, NI matrix MALDI imaging applications in positive and negative ion modes for blueberry metabolites were investigated. Crude blueberry methanol extract was utilized to compare matrix performances of 3,4-MNI, 3,6-MNI, 2,3,4-DMNI, 2,3,6-DMNI with DHB and 1,5-DAN in positive mode and with 9-AA and 1,5-DAN in negative mode (FIG. 6). In negative ion mode, 2,3,6-DMNI was the optimum matrix with intense metabolite ion signals and low matrix ion background (FIG. 6a), while in positive ion mode, 3,6-MNI was the best performing NI matrix with ion patterns similar to DHB matrix (FIG. 6b). The unique advantage of NI matrices for blueberry metabolites seemed to be in the negative ion mode 600-900 Da lipids region, where all four NI matrices demonstrate similar peak patterns with 2,3,4-DMNI the most intense, in contrast to no signal of 9-AA and noisy background of 1,5-DAN spectra, even though both matrices had good sensitivity with lipid standards (Table 1) and egg lipids extract (FIG. 5). To our knowledge, such enhancement of negative ions in 600-900 Da lipid region is unique for MALDI imaging of water-rich plant tissues. Unlike mammalian tissues, water-rich plant tissues are low in lipids with cell walls,⁸³ which could make MALDI imaging of lipids from such tissues more challenging, while reported plant lipid imaging studies were more focused on seeds (rich in lipids)^{82, 84}

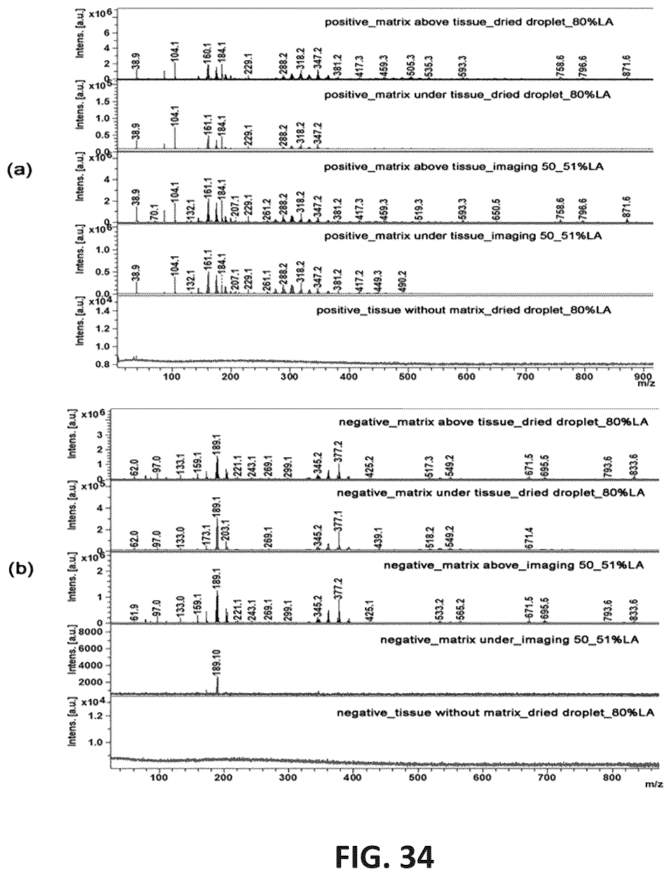

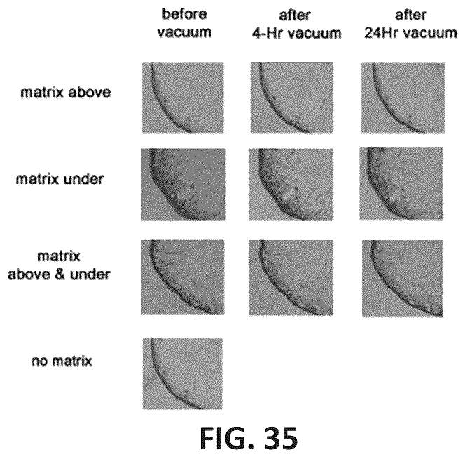

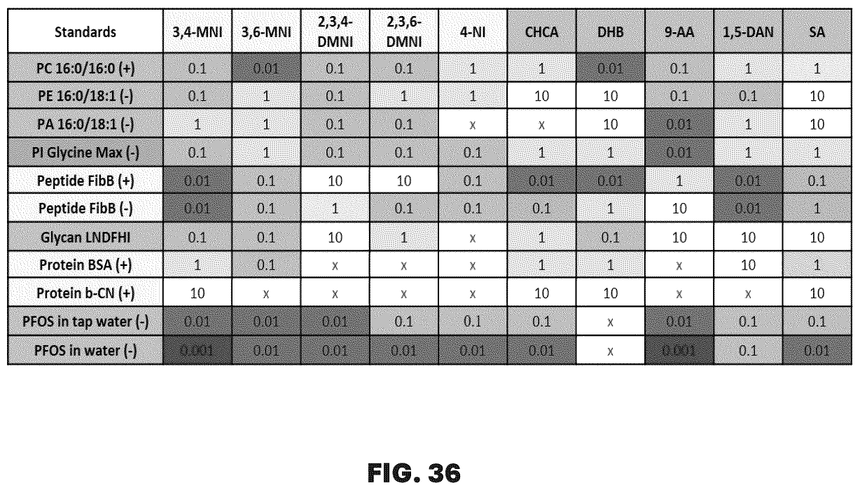

Given its superior performance for crude blueberry extract, 2,3,6-DMNI was then applied for blueberry tissue MALDI imaging. The 2,3,6-DMNI matrix formed monomer, dimer, trimer, and fragment ions at high concentrations and sublimed under the MALDI instrument vacuum (2.0 e-7 mbar) as demonstrated with QCM volatility study. To reduce matrix ion background and minimize matrix sublimation effect, frozen blueberry tissue slices were mounted onto ITO slides precoated with a thin layer of 2,3,6-DMNI matrix, then a second thin layer of matrix spray was applied over the blueberry tissue. The 2,3,6-DMNI matrix spray concentration (10 mM, 1.9 mg/mL) was much lower than the typical MALDI imaging matrix sprays, e.g. 50-150 mg/ml for DHB,^{73, 75} 15 mg/ml for DMAN.⁷³ The effectiveness of 2,3,6-DMNI matrix at low concentration could be associated to its superior laser absorption at 355 nm (FIG. 1, A_{355 nm_2,34-DMNI}=0.185, A_{355 nm_DHB}=0.0722). Applying a matrix layer under tissues shared some similarities to the surface-assisted laser desorption nano structure/nanoparticle layers^{85, 86} that were placed under the tissue layer. Control experiments (FIG. 34) performed directly on tissue without 2,3,6-DMNI matrix barely showed any signal at low (50% of absolute laser power, imaging 50 μm) or high (80% of absolute laser power, dried droplet) laser power. With the thin matrix layer above the tissue method the metabolites/lipids ion signals were intense at low and high laser powers in positive and negative ion modes, but for imaging experiments the ion intensity variation due to matrix sublimation could not be effectively compensated by the total ion counts (TIC) normalization. The thin matrix layer under the tissue method detected ion signals in positive ion mode with low and high laser power, and in negative mode with the high laser power only, suggesting that the matrix layer under tissue interacted with the laser beam effectively through the tissue thickness (40 μm) with ion desorption/ionization less efficient. Optical image comparison before and after vacuum demonstrated that the bright yellow color of the matrix applied tissue faded away due to matrix sublimation after 4-hour vacuum with the matrix layer above method, but showed little or no color change after 4-hour or 24-hour vacuum for the matrix layer under tissue method (FIG. 35), suggesting improved matrix vacuum stability. Therefore, the matrix under and above two-layer approach was chosen to combine the ion desorption advantage of the matrix layer above and the vacuum stability advantage of the matrix layer under for optimum ion images.

Blueberry metabolites were putatively identified following mass-matching and MSMS-assisted identification rationales modified from reference.⁷³ After TIC normalization, ions with higher intensity in blueberry skin/interior regions than the matrix only glass slide region (by region mean spectra and/or skyline spectra comparison) were considered promising metabolite masses, which were then compared with the blueberry crude extract Qtof ESI HRMS mass list and the matching masses were identified if within <0.05 Da error. The matched Qtof HRMS accurate masses were then searched against metabolites databases (FOODB, HMDB, LIPID MAPS) and references^{73, 75, 81, 87, 88} (<5 ppm mass accuracy typically). To further validate the assignments MALDI CID MSMS spectra were collected for blueberry extract and on tissue. The results were similar and the on-tissue CID spectra were provided in supplemental Figures SII-1 to SII-9, and compared with experimental/predicted LC-MSMS spectra in databases (FOODB, HMDB, LIPID MAPS) and/or MALDI MSMS spectra reported in references.^{73, 75, 81, 87, 88} The supplemental Excel table listed the observed masses with MALDI MS imaging and Qtof HRMS, exact masses of the putatively identified compounds, and structurally informative CID MSMS fragment ions.

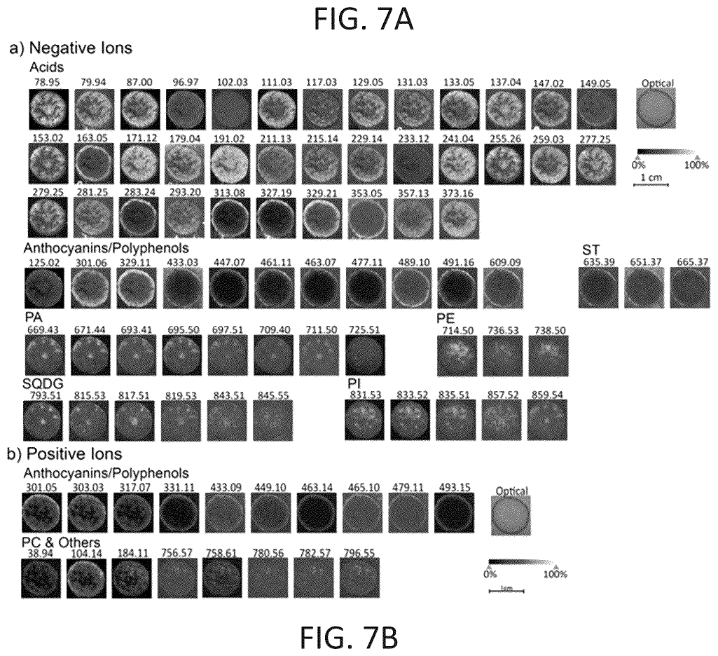

In negative ion mode, a total number of 72 metabolite ions (FIG. 7a, Figures SII-1 to SII-7, Supplemental Excel Table) were putatively identified with 2,3,6-DMNI matrix, including acids, anthocyanins/polyphenols, glycerophospholipids (PA, PE, PI), sulfoquinovosyldiacylglycerols (SQDG), and sterols (ST). The acids identified included phosphate m/z 78.95, sulfite m/z 79.94, monosaccharide phosphate m/z 259.03, aliphatic carboxylic acids (e. g. citric acid m/z 191.02, malic acid m/z 133.05, fatty acids m/z 211.13, 215.14, 229.14, 233.12, 255.26, 277.25, 279.25, 281.25, 283.24, 293.20, 313.08, 327.19, 329.21 etc.), and aromatic carboxylic acids (e. g. coumarinic acid m/z 163.05, cis-caffeic acid m/z 179.04, trans-5-O-caffeoyl-D-quinate m/z 353.05). In positive ion mode 18 metabolite ions (FIG. 7b, Figures SII-8 to SII-9, Supplemental Excel Table) were identified, e. g. potassium ions, anthocyanins/polyphenols, choline, phosphorylcholine, and PC. Although effective for glycan LNDFHI (Table 1), 2,3,6-DMNI was insensitive for shorter chain carbohydrates, and glucose or sucrose was not detected on blueberry tissue or in extract.

Overall, the 2,3,6-DMNI matrix was demonstrated very effective for blueberry metabolites/lipids identification, especially in negative ion mode and could be potentially useful for imaging applications for other plant types. The above-under tissue 2-layer matrix application method could be useful for other volatile matrices on high vacuum MALDI instrument platforms. Note that matrix vacuum stability is not an issue for atmospheric pressure MALDI (AP-MALDI) which has become commercially more available on various instrument platforms (e. g. AP-MALDI-orbitrap,⁸⁹ AP-MALDI-QQQ,⁹⁰ and iMScope™ QT⁹¹) that are useful for vacuum unstable small molecule analysis.

### CONCLUSIONS

Five nitro indole derivatives 3,4-MNI, 3,6-MNI, 2,3,4-DMNI, 2,3,6-DMNI, and 4-NI were synthesized and demonstrated to function as new dual polarity positive and negative ion MALDI matrices with broad applications. Compared to common matrices DHB, CHCA, SA, 1,5-DAN, and 9-AA, 3,4-MNI demonstrated the best overall sensitivity for all standards examined (lipid, peptide, protein, glycan, and PFOS), while other NI matrices showed high sensitivity for various compounds. The five NI matrices were synthesized as part of a larger study but are commercially available.

For complex mixtures, the best overall detection sensitivity in comparison to common matrices was demonstrated by 3,4-MNI for egg lipids extract/milk proteins/PFOS in tap water, and 2,3,6-DMNI for blueberry extract. Quantitative PFOS MALDI MS analysis with 3,4-MNI matrix demonstrated the LOQ (0.5 ppb in tap water, 0.05 ppb in MQ water, without SPE enrichment), accuracy, and precision comparable to standard LC-MS/MS MRM method or HRMS method, with additional benefits of complex mixture/salts tolerant, simple, fast, high throughput and minimal solvent consumption. With crystalline homogeneity (demonstrated by SEM and photo images) and general-purpose applications, 3,4-MNI could be potentially useful for MALDI quantitative analysis of a variety of molecules. 2,3,6-DMNI matrix was successfully applied for blueberry metabolites/lipids mapping, with a total of 90 negative or positive ions putatively identified which expanded the current knowledgebase of plant MALDI imaging.

NI matrix showed various degrees of vacuum sublimation rate under high vacuum, as measured by matrix percentage loss after 4-hr vacuum with a newly developed QCM method. However, the matrix vacuum sublimation did not affect routine MS analysis (e.g. 3,4-MNI PFOS quantitation), and a new matrix application method with thin 2,3,6-DMNI matrix layers under and above blueberry tissue allowed MALDI imaging success under high vacuum with a vacuum sublime matrix. For increasingly more available AP MALDI instrumentation, matrix vacuum stability is not an issue.

In summary, NI matrices are demonstrated to be a unique family of MALDI matrices effective in positive and negative ion mode with broad applications in many fields for qualitative and quantitative analysis. As demonstrated, slight substitution variations in indole ring structure led to distinct matrix performance changes, which indicates that nitro indole could function as a sensitive and versatile design platform for new matrix engineering to further expand applications and solve analytical challenges.

### REFERENCES FOR EXAMPLE 1

(1) Tanaka, K.; Waki, H.; Ido, Y.; Akita, S.; Yoshida, Y. Rapid Commun. Mass Spectrom. 1988, 2 (8), 151-153.
(2) Leopold, J.; Popkova, Y.; Engel, K. M.; Schiller, J. Biomolecules 2018, 8 (4), 173.
(3) Pappin, D. J.; Hojrup, P.; Bleasby, A. J. Curr. Biol. 1993, 3 (6), 327-332.
(4) Chaurand, P.; Stoeckli, M.; Caprioli, R. M. Anal. Chem. 1999, 71 (23), 5263-5270.
(5) Harvey, D. J. Mass Spectrom. Rev. 2021, 40 (4), 408-565.
(6) McLuckey, S. A.; Van Berkel, G. J.; Glish, G. L. J. Am. Soc. Mass. Spectrom. 1992, 3 (1), 60-70.
(7) Montaudo, G.; Samperi, F.; Montaudo, M. S. Prog. Polym. Sci. 2006, 31 (3), 277-357.
(8) Zenobi, R.; Knochenmuss, R. Mass Spectrom. Rev. 1998, 17 (5), 337-366.
(9) Knochenmuss, R.; Zenobi, R. Chem. Rev. 2003, 103 (2), 441-452.
(10) Calvano, C. D.; Ventura, G.; Cataldi, T. R.; Palmisano, F. Anal. Bioanal. Chem. 2015, 407 (21), 6369-6379.
(11) WeiBflog, J.; Svatos, A. RSC Adv. 2016, 6 (79), 75073-75081.
(12) Scott, A. J.; Flinders, B.; Cappell, J.; Liang, T.; Pelc, R. S.; Tran, B.; Kilgour, D. P.; Heeren, R. M.; Goodlett, D. R.; Ernst, R. K. FEMS Pathog. Dis. 2016, 74 (8), ftw097.
(13) Dong, W.; Shen, Q.; Baibado, J. T.; Liang, Y.; Wang, P.; Huang, Y.; Zhang, Z.; Wang, Y.; Cheung, H.-Y. Int. J. Mass spectrom. 2013, 343, 15-22.
(14) Thomas, A. I.; Charbonneau, J. L.; Fournaise, E.; Chaurand, P. Anal. Chem. 2012, 84 (4), 2048-2054.
(15) Li, B.; Sun, R.; Gordon, A.; Ge, J.; Zhang, Y.; Li, P.; Yang, H. Anal. Chem. 2019, 91 (13), 8221-8228.
(16) Huang, P.; Huang, C.-Y.; Lin, T.-C.; Lin, L.-E.; Yang, E.; Lee, C.; Hsu, C.-C.; Chou, P.-T. Anal. Chem. 2020, 92 (10), 7139-7145.
(17) Tang, W.; Gordon, A.; Wang, F.; Chen, Y.; Li, B. Anal. Chem. 2021, 93 (26), 9083-9093.
(18) Kaushik, N. K.; Kaushik, N.; Attri, P.; Kumar, N.; Kim, C. H.; Verma, A. K.; Choi, E. H. Molecules 2013, 18 (6), 6620-6662.
(19) Nielen, M. W.; Malucha, S. Rapid Commun. Mass Spectrom. 1997, 11 (11), 1194-1204.
(20) Galesio, M.; Rial-Otero, R.; Capelo-Martinez, J. L. Rapid Commun. Mass Spectrom. 2009, 23 (12), 1783-1791.
(21) Habumugisha, T.; Zhang, Z.; Ndayishimiye, J. C.; Nkinahamira, F.; Kayiranga, A.; Cyubahiro, E.; Rehman, A.; Yan, C.; Zhang, X. Anal. Methods 2022, 14 (7), 763-772.
(22) Bai, J.; Liang, X.; Liu, Y. H.; Zhu, Y.; Lubman, D. M. Rapid Commun. Mass Spectrom. 1996, 10 (7), 839-844.
(23) Nonami, H.; Fukui, S.; Erra-Balsells, R. J. Mass Spectrom. 1997, 32 (3), 287-296.
(24) Nonami, H.; Tanaka, K.; Fukuyama, Y.; Erra-Balsells, R. Rapid Commun. Mass Spectrom. 1998, 12 (6), 285-296.
(25) Nonami, H.; Wu, F.; Thummel, R. P.; Fukuyama, Y.; Yamaoka, H.; Erra-Balsells, R. Rapid Commun. Mass Spectrom. 2001, 15 (23), 2354-2373.
(26) Yamagaki, T.; Nakanishi, H. Glycoconj. J. 1999, 16 (8), 385-389.
(27) Nuutinen, J. M.; Purmonen, M.; Ratilainen, J.; Rissanen, K.; Vainiotalo, P. Rapid Commun. Mass Spectrom. 2001, 15 (15), 1374-1381.
(28) Lloyd, P. M.; Suddaby, K. G.; Varney, J. E.; Scrivener, E.; Derrick, P. J.; Haddleton, D. M. Eur. Mass Spectrom. 1995, 1 (3), 293-300.
(29) Choi, H.; Lee, D.; Kim, Y.; Nguyen, H.-Q.; Han, S.; Kim, J. J. Am. Soc. Mass. Spectrom. 2019, 30 (7), 1174-1178.
(30) Asakawa, D.; Smargiasso, N.; De Pauw, E. Anal. Chem. 2012, 84 (17), 7463-7468.
(31) Fukuyama, Y.; Izumi, S.; Tanaka, K. J. Am. Soc. Mass. Spectrom. 2018, 29 (11), 2227-2236.
(32) Fukuyama, Y.; Izumi, S.; Tanaka, K. Anal. Chem. 2016, 88 (16), 8058-8063.
(33) Steven, R. T.; Race, A. M.; Bunch, J. J. Am. Soc. Mass. Spectrom. 2013, 24 (5), 801-804.
(34) Bonesi, S. M.; Mesaros, M.; Cabrerizo, F. M.; Ponce, M. A.; Bilmes, G. M.; Erra-Balsells, R. Chem. Phys. Lett. 2007, 446 (1-3), 49-55.
(35) Tarzi, O. I.; Ponce, M. A.; Cabrerizo, F. M.; Bonesi, S. M.; Erra-Balsells, R. Arkivoc 2005, 12, 295-310.
(36) Hadida Ruah, S. S.; Grootenhuis, P. D. J.; Van Goor, F. F.; Zhou, J.; Bear, B. R.; Miller, M. T.; McCartney, J.; Djamel Numa, M. M.; Yang, X.; Nair, N. US 20160271105A1, Sep. 22, 2016.
(37) https://foodb.ca/
(38) https://hmdb.ca/
(39) Fahy, E.; Sud, M.; Cotter, D.; Subramaniam, S. Nucleic Acids Res. 2007, 35 (suppl_2), W606-W612.
(40) Balasanmugam, K.; Viswanadham, S. K.; Hercules, D. M. Anal. Chem. 1983, 55 (14), 2424-2426.
(41) Awad, H.; Stoudemayer, M.; Usher, L.; Amster, I.; Cohen, A.; Das, U.; Whittal, R.; Dimmock, J.; EI-Aneed, A. J. Mass Spectrom. 2014, 49 (11), 1139-1147.
(42) Kang, C.; Zhou, Y.; Du, Z.; Bian, Z.; Wang, J.; Qiu, X.; Gao, L.; Sun, Y. J. Mass Spectrom. 2013, 48 (12), 1318-1324.
(43) Yang, J.; Norris, J. L.; Caprioli, R. J. Mass Spectrom. 2018, 53 (10), 1005-1012.
(44) O'sullivan, C.; Guilbault, G. Biosens. Bioelectron. 1999, 14 (8-9), 663-670.
(45) Quist, A. P.; Huth-Fehre, T.; Sundqvist, B. U.; Vertes, A. Rapid Commun. Mass Spectrom. 1994, 8 (2), 149-154.
(46) Wallace, W. E.; Arnould, M.; Knochenmuss, R. Int. J. Mass Spectrom. 2005, 242 (1), 13-22.
(47) Jahnke, A.; Berger, U. J. Chromatogr. A 2009, 1216 (3), 410-421.
(48) Naidu, R.; Nadebaum, P.; Fang, C.; Cousins, I.; Pennell, K.; Conder, J.; Newell, C.; Longpre, D.; Warner, S.; Crosbie, N. Environ. Technol. Innov. 2020, 19, 100915.
(49) Yang, W.; Guo, J.; Dong, L.; Huang, Y. https://www.agilent.com/cs/library/applications/application-environmental-pfo-and-pfoa-ultivo-Ictq-5994-0437en-agilent.pdf (50) Xiang, L.; Sun, T.-F.; Chen, L.; Xiao, T.; Cai, Q.-Y.; Li, H.; He, D.-C.; Wong, M.-H.; Li, Y.-W.; Mo, C.-H. Food Anal. Methods 2017, 10(7), 2518-2528.
(51) Mullin, L.; Cleland, G. https://www.waters.com/webassets/cms/library/docs/720005727en. pdf
(52) Haghani, A.; Eaton, A.; Eaton, E.; CA, M.; Jack, R.; Bromirski, M. https://assets.thermofisher.com/TFS-Assets/CMD/Application-Notes/an-65499-Ic-ms-epa-method-537-1-validation-an65499-en.pdf
(53) UIIah, S.; Alsberg, T.; Vestergren, R.; Berger, U. Anal. Bioanal. Chem. 2012, 404, 2193-2201.
(54) Al Amin, M.; Sobhani, Z.; Liu, Y.; Dharmaraja, R.; Chadalavada, S.; Naidu, R.; Chalker, J. M.; Fang, C. Environ. Technol. Innov. 2020, 19, 100879.
(55) Li, X.; Li, T.; Wang, Z.; Wei, J.; Liu, J.; Zhang, Y.; Zhao, Z. Talanta 2021, 226, 122150.
(56) Bian, Y.; He, M.-Y.; Ling, Y.; Wang, X.-J.; Zhang, F.; Feng, X.-S.; Zhang, Y.; Xing, S.-G.; Li, J.; Qiu, X. Environ. Pollut. 2022, 293, 118505.
(57) Cao, D.; Wang, Z.; Han, C.; Cui, L.; Hu, M.; Wu, J.; Liu, Y.; Cai, Y.; Wang, H.; Kang, Y. Talanta 2011, 85 (1), 345-352.
(58) Kawasaki, H.; Shimomae, Y.; Watanabe, T.; Arakawa, R. Colloids Surf. A 2009, 347 (1-3), 220-224.
(59) Haug, A.; Hostmark, A. T.; Harstad, O. M. Lipids Health Dis. 2007, 6 (1), 1-16.
(60) Vincent, D.; Elkins, A.; Condina, M. R.; Ezernieks, V.; Rochfort, S. PLoS One 2016, 11 (10), e0163471.
(61) Di Francesco, L.; Di Girolamo, F.; Mennini, M.; Masotti, A.; Salvatori, G.; Rigon, G.; Signore, F.; Pietrantoni, E.; Scapaticci, M.; Lante, I. Nutrients 2018, 10 (9), 1238.
(62) Sun, G.; Yang, K.; Zhao, Z.; Guan, S.; Han, X.; Gross, R. W. Anal. Chem. 2008, 80 (19), 7576-7585.
(63) Harvey, D. J. J. Mass Spectrom. 1995, 30 (9), 1333-1346.
(64) Teuber, K.; Schiller, J.; Fuchs, B.; Karas, M.; Jaskolla, T. W. Chem. Phys. Lipids 2010, 163 (6), 552-560.
(65) Wood, P. L.; Muir, W.; Christmann, U.; Gibbons, P.; Hancock, C. L.; Poole, C. M.; Emery, A. L.; Poovey, J. R.; Hagg, C.; Scarborough, J. H. Poult. Sci. 2021, 100 (2), 887-899.
(66) Zhou, L.; Wang, Y.; Wang, X.; Liang, Y.; Huang, Z.; Zeng, X. J. Agric. Food Chem. 2017, 65 (6), 1229-1238.
(67) Wang, Z.; Cai, Y.; Wang, Y.; Zhou, X.; Zhang, Y.; Lu, H. Sci. Rep. 2017, 7 (1), 1-9.
(68) Hsu, F.-F.; Turk, J. J. Chromatogr. B 2009, 877 (26), 2673-2695.
(69) https://www.lipidmaps.orq/resources/standards.php
(70) https://www.lipidmaps.orq/resources/tools/ms
(71) Ekroos, K.; Ejsing, C. S.; Bahr, U.; Karas, M.; Simons, K.; Shevchenko, A. J. Lipid Res. 2003, 44 (11), 2181-2192.
(72) Zhang, W.; Jian, R.; Zhao, J.; Liu, Y.; Xia, Y. J. Lipid Res. 2022, 100219.
(73) Ye, H.; Gemperline, E.; Venkateshwaran, M.; Chen, R.; Delaux, P. M.; Howes-Podoll, M.; Ane, J. M.; Li, L. Plant J. 2013, 75 (1), 130-145.
(74) Sturtevant, D.; Lee, Y.-J.; Chapman, K. D. Curr. Opin. Biotechnol. 2016, 37, 53-60.
(75) Yoshimura, Y.; Enomoto, H.; Moriyama, T.; Kawamura, Y.; Setou, M.; Zaima, N. Anal. Bioanal. Chem. 2012, 403 (7), 1885-1895.
(76) Wang, J.; Yang, E.; Chaurand, P.; Raghavan, V. Food Chem. 2021, 345, 128838.
(77) Enomoto, H.; Sato, K.; Miyamoto, K.; Ohtsuka, A.; Yamane, H. J. Agric. Food Chem. 2018, 66 (19), 4958-4965.
(78) Nakamura, J.; Morikawa-Ichinose, T.; Fujimura, Y.; Hayakawa, E.; Takahashi, K.; Ishii, T.; Miura, D.; Wariishi, H. Anal. Bioanal. Chem. 2017, 409, 1697-1706.
(79) Li, B.; Neumann, E. K.; Ge, J.; Gao, W.; Yang, H.; Li, P.; Sweedler, J. V. Plant Cell Environ. 2018, 41 (11), 2693-2703.
(80) Enomoto, H. Molecules 2020, 25 (20), 4600.
(81) Dare, A. P.; Gunther, C. S.; Grey, A. C.; Guo, G.; Demarais, N. J.; Cordiner, S.; McGhie, T. K.; Boldingh, H.; Hunt, M.; Deng, C. Food Chem. 2022, 374, 131703.
(82) Shi, Y.; Hu, H.; Hao, Q.; Wu, R.; Wang, L.; Qin, L.; Gu, W.; Liu, H.; Jiang, D.; Hong, L., Chem. Commun. 2022, 58 (5), 633-636.
(83) Liu, H.; Pan, Y.; Xiong, C.; han, J.; Wang, X.; Chen, J.; Nie, Z. TrAC, Trends Anal. Chem. 2022, 157, 116809.
(84) de Souza, L. P.; Borghi, M.; Fernie, A., Int. J. Mol. Sci. 2020, 21 (23), 8987.
(85) Greving, M. P.; Patti, G. J.; Siuzdak, G. Anal. Chem. 2011, 83 (1), 2-7.
(86) Iakab, S.-A.; Baquer, G.; Lafuente, M.; Pina, M. P.; Ramirez, J. L.; Rafols, P.; Correig-Blanchar, X.; Garcia-Altares, M. Anal. Chem. 2022, 94 (6), 2785-2793.
(87) Bederska-Lojewska, D.; Pieszka, M.; Marzec, A.; Rudzinska, M.; Grygier, A.; Siger, A.; Cieslik-Boczula, K.; Orczewska-Dudek, S.; Migdaf, W. Molecules 2021, 26 (24), 7446.
(88) Forney, C. F.; Kalt, W.; Jordan, M. A.; Vinqvist-Tymchuk, M. R.; Fillmore, S. A. J. Berry Res. 2012, 2 (3), 169-177.
(89) Mahale, V.; Singh, A.; Phadke, G. S.; Ghanate, A. D.; Oulkar, D. P.; Banerjee, K.; Panchagnula, V. J. AOAC Int. 2017, 100 (3), 640-646.
(90) Bowman, A. P.; Sawicki, J.; Talaty, N. N.; Buck, W. R.; Yang, J.; Wagner, D. S. Pharm. 2022, 15 (10), 1180.
(91) Islam, A.; Sakamoto, T.; Zhai, Q.; Rahman, M. M.; Mamun, M. A.; Takahashi, Y.; Kahyo, T.; Setou, M. Pharm. 2022, 15 (11), 1314.

TABLE S1
QCM NI matrices vacuum stability (percentage of loss) measurements

| Matrix | Trial_1 | Trial_2 | Trial_3 | Average | RSD % |
|---|---|---|---|---|---|
| 3,4-MNI | 78.58% | 72.89% | 78.13% | 76.54% | 3.4% |
| 3,6-MNI | 80.43% | 79.13% | 73.93% | 77.83% | 3.6% |
| 2,3,4-DMNI | 30.87% | 55.51% | 14.85% | 33.74% | 49.6% |
| 2,3,6-DMNI | 51.43% | 64.54% | 43.50% | 53.16% | 16.3% |
| 4-NI | 21.0% | 34.94% | 32.69% | 29.54% | 20.7% |

TABLE S2-1
PFOS in MQ water calibration standard (CS) and verification
sample (Sv) MALDI response statistical results

| | | | PFOS/IS | 499/507_ave | |
|---|---|---|---|---|---|
| | PFOS | IS | Conc. | Peak area | RSD |
| Sample | (ppb) | (ppb) | ratio | ratio | % |
| CS1 | 0.05 | 10 | 0.005 | 0.0076 | 8% |
| CS2 | 0.1 | 10 | 0.01 | 0.0139 | 5% |
| CS3 | 0.5 | 10 | 0.05 | 0.0621 | 4% |
| CS4 | 1 | 10 | 0.1 | 0.1558 | 6% |
| CS5 | 2 | 10 | 0.2 | 0.2188 | 5% |
| CS6 | 10 | 10 | 1 | 1.2677 | 4% |
| | | | | Average RSD | 5% |
| Sv | 5 | 10 | 0.5 | 0.6115 | 4% |

TABLE S2-2
PFOS in tap water calibration standard (CS) and verification
sample (Sv) MALDI response statistical results

| | | | PFOS/IS | 499/507_ave | |
|---|---|---|---|---|---|
| | PFOS | IS | conc. | Peak area | RSD |
| Sample | (ppb) | (ppb) | ratio | ratio | % |
| CS1 | 0.5 | 10 | 0.05 | 0.0819 | 10% |
| CS2 | 1 | 10 | 0.1 | 0.1085 | 2% |
| CS3 | 2 | 10 | 0.2 | 0.2403 | 8% |
| CS4 | 10 | 10 | 1 | 1.1975 | 1% |
| CS5 | 20 | 10 | 2 | 2.5943 | 2% |
| CS6 | 50 | 10 | 5 | 7.1005 | 1% |
| | | | | Average RSD | 4% |
| Sv | 5 | 10 | 0.50 | 0.6562 | 3% |

TABLE S3
Egg lipids assigned peaks in positive and negative ion
spectra acquired with 3,4-MNI matrix

| Posi- | | Fatty | Posi- | | Fatty |
|---|---|---|---|---|---|
| tive | Assign- | acid | tive | Assign- | acid |
| ions | ment | moieties | ions | ment | moieties |
| 703.6 | SM 16:0 + H | 16:0 | 806.6 | PC 38:6 + H | 18:2; 20:4 |
| 725.6 | SM 16:0 + | 16:0 | 806.6 | PC 36:3 + | 18:1; 18:2 |
| | Na | | | Na | |
| 758.6 | PC 34:2 + H | 16:0; 18:2 | 808.6 | PC 38:5 + H | 18:1; 20:4 |
| 760.6 | PC 34:1 + H | 16:0; 18:1 | 808.6 | PC 36:2 + | 18:0; 18:2 |
| | | | | Na | |
| 780.6 | PC 34:2 + Na | 16:0; 18:2 | 810.6 | PC 38:4 + H | 18:0; 20:4 |
| 782.6 | PC 34:1 + Na | 16:0; 18:1 | 810.6 | PC 36:1 + | 18:0; 18:1 |
| | | | | Na | |
| 786.6 | PC 36:2 + H | 18:0; 18:2 | 824.6 | PC 36:2 + K | 18:0; 18:2 |
| 788.6 | PC 36:1 + H | 18:0; 18:1 | 826.6 | PC 36:1 + K | 18:0; 18:1 |
| 796.6 | PC 34:2 + K | 16:0; 18:2 | | | |
| 798.6 | PC 34:1 + K | 16:0; 18:1 | | | |
| Neg- | Assign- | Fatty | Neg- | Assign- | Fatty |
| ative | ment | acid | ative | ment | acid |
| ions | [M − H]⁻ | moieties | ions | [M − H]⁻ | moieties |
| 671.5 | PA 34:2 | 16:0; 18:2 | 790.5 | PE 40:6 | 18:0; 22:6 |
| 673.5 | PA 34:1 | 16:0; 18:1 | 792.5 | PE 40:5 | 18:0; 22:5 |
| 687.5 | PA 35:1 | 17:0; 18:1; | 794.5 | PE 40:4 | 18:0; 22:4 |
| | | 16:0; 19:1 | | | |
| 697.5 | PA 36:3 | 18:1; 18:2 | 833.5 | PI 34:2 | 16:0; 18:2 |
| | | 16:0; 20:3 | | | |
| 699.5 | PA 36:2 | 18:1; 18:1 | 835.5 | PI 34:1 | 16:0; 18:1 |
| | | 16:0; 20:2 | | | |
| 714.5 | PE 34:2 | 16:0; 18:2 | 857.5 | PI 36:4 | 16:0; 20:4; |
| | | | | | 18:2; 18:2 |
| 716.5 | PE 34:1 | 16:0; 18:1 | 859.5 | PI 36:3 | 16:0; 20:3; |
| | | | | | 18:1; 18:2 |
| 738.5 | PE 36:4 | 18:2; 18:2 | 861.5 | PI 36:2 | 16:0; 20:2; |
| | | 16:0; 20:4 | | | 18:0; 18:2 |
| 740.5 | PE 36:3 | 18:1; 18:2 | 885.5 | PI 38:4 | 18:0; 20:4 |
| | | 16:0; 20:3 | | | |
| 742.5 | PE 36:2 | 18:2, 18:0 | 887.6 | PI 38:3 | 18:0; 20:3 |
| | | 16:0; 20:2 | | | |
| 744.5 | PE 36:1 | 18:0; 18:1 | 911.6 | PI 40:5 | 18:0; 22:5; |
| | | | | | 20:1; 20:4 |
| 762.5 | PE 38:6 | 20:4; 18:2 | 913.6 | PI 40:4 | 18:0; 22:4; |
| | | 16:0; 22:6 | | | 20:1; 20:3 |
| 764.5 | PE 38:5 | 18:1; 20:4 | | | |
| | | 16:0; 22:5 | | | |
| 766.5 | PE 38:4 | 18:0; 20:4 | | | |
| 768.5 | PE 38:3 | 18:0; 20:3 | | | |

### Example 2

Bulk-Scale Nitration of Indole Substrates. The bulk nitration reaction of all the indole substrates using [(Por)Fe^{III}(O₂^{-●})] and NO_{(g)} was carried out by a generalized procedure as follows: A 100 mL Schlenk flask containing [(TPP)Fe^{III}] (200 mg, 0.3 mmol) in THF (25 mL) was cooled down in an acetone/liquid N₂ bath adjusted to −80° C. Upon temperature equilibration, dioxygen gas (or labeled¹⁸O_{2(g)}) was bubbled through to form [(TPP)Fe^{III}(O₂^{-●})]. Then, the indole substrate (0.3 mmol; in 2 mL of THF) was added to it, and subsequently, 2 mL of NO_{(g)} (or labelled ¹⁵NO_{(g)}) was bubbled into the solution at −80° C. using a three-way gastight syringe. The solution was stirred at −80° C. for 30 minutes before warming to room temperature and immediately concentrated in vacuum. The final nitrated products (4-, 6-, and N-nitro 2,3 dimethylindole) were purified by silica gel column chromatography using DCM:hexane as an eluent, and characterized by ¹H and ¹³C NMR, IR, ESI-MS and LC-MS methodologies.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1 percent to about 5 percent” should be interpreted to include not only the explicitly recited concentration of about 0.1 weight percent to about 5 weight percent but also include individual concentrations (e.g., 1 percent, 2 percent, 3 percent, and 4 percent) and the sub-ranges (e.g., 0.5 percent, 1.1 percent, 2.2 percent, 3.3 percent, and 4.4 percent) within the indicated range. The term “about” can include traditional rounding according to significant figures of the numerical value. In addition, the phrase “about ‘x’ to ‘y’” includes “about ‘x’ to about ‘y’”. Many variations and modifications may be made to the above-described aspects. All such modifications and variations are intended to be included herein within the scope of this disclosure and protected by the following claims.

What is claimed:

1. A matrix material for Matrix Assisted Laser Desorption/Ionization (MALDI) comprising at least one MALDI matrix compound having a formula represented by the following structure: 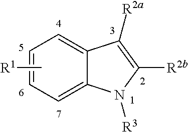
wherein
each R¹ is independently selected from hydrogen, an alkyl group, an alkenyl group, an alkoxyl group, a carbonyl group, a carboxyl group, a primary amine, a secondary amine, a tertiary amine, an ether, halogen, hydroxy, cyano, thiol, and trifluoromethyl;
R^{2a} and R^{2b} are independently selected from hydrogen, an alkyl group, an alkenyl group, an alkoxyl group, a carbonyl group, a carboxyl group, a primary amine group, a secondary amine group, a tertiary amine group, an ether group, halogen, hydroxy group, cyano group, thiol group, trifluoromethyl group, and a substituted or unsubstituted indole group; and
R³ is hydrogen or a substituted or unsubstituted indole group.

2. The matrix material of claim 1, wherein at least one R¹ is nitro.

3. The matrix material of claim 1, wherein at least one of R^{2a} and R^{2b} is hydrogen.

4. The matrix material of claim 1, wherein
each R¹ are independently selected from hydrogen, a C1 to C6 alkyl group, a carbonyl group, a carboxyl group, nitro group, thiol group, a primary amine group, a secondary amine group, or a tertiary amine group;
R^{2a} and R^{2b} are independently selected from hydrogen, a C1 to C6 alkyl group, a carbonyl group, a carboxyl group, nitro group, thiol group, a primary amine group, a secondary amine group, a tertiary amine group, or a substituted or unsubstituted indole group; and
R³ is hydrogen or a substituted or unsubstituted indole group.

5. The matrix material of claim 1, wherein the material comprises a substituted or unsubstituted indole dimer, indole trimer, indole tetramer, indole pentamer, or indole hexamer.

6. The matrix material of claim 1, comprising a at least one MALDI matrix compound having a formula represented by any one of the following structures:

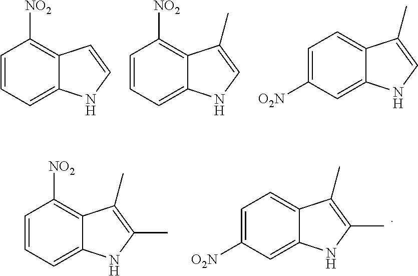

7. A method for providing a plurality of analyte ions for Matrix Assisted Laser Desorption/Ionization (MALDI) mass spectrometry comprising:
providing a matrix material comprising the at least one MALDI matrix compound of claim 1;
depositing at least the matrix mixture and an analyte onto a support forming a matrix and analyte surface;
irradiating the matrix and analyte surface to desorb and ionize at least part of the analyte into the gas-phase, forming a plurality of gas-phase ions; and
separating the ionized analyte molecules based on their mass-to-charge ratio (m/z) using a mass analyzer.

8. The method of claim 7, wherein the forming the matrix and analyte surface is selected from:
mixing at least the matrix material and analyte together to form a first mixture and depositing the first mixture onto the support;
spotting at least the matrix material and the analyte in layers onto the support;
spraying the matrix material onto the support, wherein the analyte can be deposited before or after spraying;
subliming the matrix material onto the support, wherein the analyte can be deposited before or after spraying; and
any combination thereof.

9. The method of claim 7, wherein the analyte is deposited onto the support in the form of a solution, suspension, solid particles, or surface layers.

10. The method of claim 7, further including depositing onto the support at least one additive.

11. The method of claim 10, wherein the additive is part of the initial matrix material.

12. The method of claim 10, wherein the additive is a salt, a buffer, a small molecule, or any combination thereof.

13. The method of claim 7, wherein the mass analyzer is selected from the group consisting of time of flight, quadrupole, magnetic sector, ion trap, orbitrap, fourier transform ion cyclotron resonance, or any combination thereof.

14. The method of claim 7, further comprising using a buffer gas to separate the gas-phase ions.

15. The method of claim 7, wherein the analyte comprises a biomolecule.

16. The method of claim 7, wherein the analyte is an amino acid, a lipid, a peptide, a protein, a glycan, a carbohydrate, an oligonucleotide, a metabolite, an environmental contaminant, a pharmaceutical, a polymer, or any combination thereof.

17. A method of nitrating indole, comprising:
cooling a mixture comprising an iron porphyrin complex and a solvent;
bubbling dioxygen gas through the mixture;
adding an indole substrate to the mixture; and
bubbling nitric oxide gas into the mixture.

18. The method of claim 17, wherein the iron porphyrin complex is (TPP)Fe^{III}. The method of claim 8, wherein the solvent is THF or dichloromethane.

19. A compound produced by the method of claim 17.
